(19) United States
(12) Patent Application Publication
(10) Pub. No.: US 2021/0355129 A1
(43) Pub. Date: Nov. 18, 2021

(54) NOVEL UREA 6,7-DIHYDRO-4H-PYRAZOLO[1,5-A]PYRAZINES ACTIVE AGAINST THE HEPATITIS B VIRUS (HBV)

(71) Applicant: AICURIS GMBH & CO. KG, Wuppertal (DE)

(72) Inventors: Alastair DONALD, Wuppertal (DE); Andreas URBAN, Sprockhövel (DE); Susanne BONSMANN, Köln (DE)

(73) Assignee: AICURIS GMBH & CO. KG, Wuppertal (DE)

(21) Appl. No.: 17/290,417

(22) PCT Filed: Nov. 1, 2019
(86) PCT No.: PCT/EP2019/079977

§ 371 Date: Apr. 30, 2021

(30) Foreign Application Priority Data
Nov. 2, 2018 (EP) 18000877.3

(51) Int. Cl. C07D 487/04; A61P 31/12

(52) U.S. Cl. CPC C07D 487/04; A61P 31/12

(57) ABSTRACT

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes and intermediates for making the compounds.

### TECHNICAL FIELD

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes for making the compounds.

### BACKGROUND OF THE INVENTION

Chronic HBV infection is a significant global health problem, affecting over 5% of the world population (over 350 million people worldwide and 1.25 million individuals in the US). Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact.

The low cure rates of HBV are attributed at least in part to the fact that complete suppression of virus production is difficult to achieve with a single antiviral agent, and to the presence and persistence of covalently closed circular DNA (cccDNA) in the nucleus of infected hepatocytes. However, persistent suppression of HBV DNA slows liver disease progression and helps to prevent hepatocellular carcinoma (HCC).

Current therapy goals for HBV-infected patients are directed to reducing serum HBV DNA to low or undetectable levels, and to ultimately reducing or preventing the development of cirrhosis and HCC.

The HBV is an enveloped, partially double-stranded DNA (dsDNA) virus of the hepadnavirus family (Hepadnaviridae). HBV capsid protein (HBV-CP) plays essential roles in HBV replication. The predominant biological function of HBV-CP is to act as a structural protein to encapsidate pre-genomic RNA and form immature capsid particles, which spontaneously self-assemble from many copies of capsid protein dimers in the cytoplasm.

HBV-CP also regulates viral DNA synthesis through differential phosphorylation states of its C-terminal phosphorylation sites. Also, HBV-CP might facilitate the nuclear translocation of viral relaxed circular genome by means of the nuclear localization signals located in the arginine-rich domain of the C-terminal region of HBV-CP.

In the nucleus, as a component of the viral cccDNA mini-chromosome, HBV-CP could play a structural and regulatory role in the functionality of cccDNA mini-chromosomes. HBV-CP also interacts with viral large envelope protein in the endoplasmic reticulum (ER), and triggers the release of intact viral particles from hepatocytes.

HBV-CP related anti-HBV compounds have been reported. For example, phenylpropenamide derivatives, including compounds named AT-61 and AT-130 (Feld J. et al. Antiviral Res. 2007, 76, 168), and a class of thiazolidin-4-ones from Valeant (WO2006/033995), have been shown to inhibit pre-genomic RNA (pgRNA) packaging.

F. Hoffmann-LA Roche AG have disclosed a series of 3-substituted tetrahydro-pyrazolo[1,5-a]pyrazines for the therapy of HBV (WO2016/113273, WO2017/198744, WO2018/011162, WO2018/011160, WO2018/011163).

Heteroaryldihydropyrimidines (HAPs) were discovered in a tissue culture-based screening (Weber et al., Antiviral Res. 2002, 54, 69). These HAP analogs act as synthetic allosteric activators and are able to induce aberrant capsid formation that leads to degradation of HBV-CP (WO 99/54326, WO 00/58302, WO 01/45712, WO 01/6840). Further HAP analogs have also been described (J. Med. Chem. 2016, 59 (16), 7651-7666).

A subclass of HAPs from F. Hoffman-La Roche also shows activity against HBV (WO2014/184328, WO2015/132276, and WO2016/146598). A similar subclass from Sunshine Lake Pharma also shows activity against HBV (WO2015/144093). Further HAPs have also been shown to possess activity against HBV (WO2013/102655, Bioorg. Med. Chem. 2017, 25(3) pp. 1042-1056, and a similar subclass from Enanta Therapeutics shows similar activity (WO2017/011552). A further subclass from Medshine Discovery shows similar activity (WO2017/076286). A further subclass (Janssen Pharma) shows similar activity (WO2013/102655).

A subclass of pyridazones and triazinones (F. Hoffman-La Roche) also show activity against HBV (WO2016/023877), as do a subclass of tetrahydropyridopyridines (WO2016/177655). A subclass of tricyclic 4-pyridone-3-carboxylic acid derivatives from Roche also show similar anti-HBV activity (WO2017/013046).

A subclass of sulfamoyl-arylamides from Novira Therapeutics (now part of Johnson & Johnson Inc.) also shows activity against HBV (WO2013/006394, WO2013/096744, WO2014/165128, WO2014/184365, WO2015/109130, WO2016/089990, WO2016/109663, WO2016/109684, WO2016/109689, WO2017/059059). A similar subclass of thioether-arylamides (also from Novira Therapeutics) shows activity against HBV (WO2016/089990). Additionally, a subclass of aryl-azepanes (also from Novira Therapeutics) shows activity against HBV (WO2015/073774). A similar subclass of arylamides from Enanta Therapeutics show activity against HBV (WO2017/015451).

Sulfamoyl derivatives from Janssen Pharma have also been shown to possess activity against HBV (WO2014/033167, WO2014/033170, WO2017001655, J. Med. Chem, 2018, 61(14) 6247-6260)

A subclass of glyoxamide substituted pyrrolamide derivatives also from Janssen Pharma have also been shown to possess activity against HBV (WO2015/011281). A similar class of glyoxamide substituted pyrrolamides (Gilead Sciences) has also been described (WO2018/039531).

A subclass of sulfamoyl- and oxalyl-heterobiaryls from Enanta Therapeutics also show activity against HBV (WO2016/161268, WO2016/183266, WO2017/015451, WO2017/136403 & US20170253609).

A subclass of aniline-pyrimidines from Assembly Biosciences also show activity against HBV (WO2015/057945, WO2015/172128). A subclass of fused tri-cycles from Assembly Biosciences (dibenzo-thiazepinones, dibenzo-diazepinones, dibenzo-oxazepinones) show activity against HBV (WO2015/138895, WO2017/048950).

A series of cyclic sulfamides has been described as modulators of HBV-CP function by Assembly Biosciences (WO2018/160878).

A series of cyclic sulfamides has been described as modulators of HBV-CP function by Assembly Biosciences (WO2018/160878).

Arbutus Biopharma have disclosed a series of benzamides for the therapy of HBV (WO2018/052967, WO2018/172852).

It was also shown that the small molecule bis-ANS acts as a molecular ‘wedge’ and interferes with normal capsid-protein geometry and capsid formation (Zlotnick A et al. J. Virol. 2002, 4848).

Problems that HBV direct acting antivirals may encounter are toxicity, mutagenicity, lack of selectivity, poor efficacy, poor bioavailability, low solubility and difficulty of synthesis. There is a thus a need for additional inhibitors for the treatment, amelioration or prevention of HBV that may overcome at least one of these disadvantages or that have additional advantages such as increased potency or an increased safety window.

Administration of such therapeutic agents to an HBV infected patient, either as monotherapy or in combination with other HBV treatments or ancillary treatments, will lead to significantly reduced virus burden, improved prognosis, diminished progression of the disease and/or enhanced seroconversion rates.

### SUMMARY OF THE INVENTION

Provided herein are compounds useful for the treatment or prevention of HBV infection in a subject in need thereof, and intermediates useful in their preparation. The subject matter of the invention is a compound of Formula I

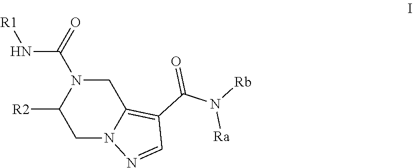

in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O-C1-C6-alkyl, optionally substituted with 1, 2, or 3 groups each independently selected from OH, halo, carboxy, C3-C7-heterocycloalkyl, C1-C6-alkyl, C1-C6-haloalkyl, C1-C6-hydroxyalkyl, C1-C6-alkyl-O-C1-C6-alkyl, C1-C6-alkyl-O-C1-C6-halo alkyl C1-C6-alkyl-S-C1-C6-alkyl, C1-C6-alkyl-SO₂-C1-C6-alkyl, C1-C6-alkyl-C≡N, C1-C2-alkyl-O-C3-C6-cycloalkyl, C1-C2-alkyl-C3-C7-heterocyclo alkyl, C1-C2-alkyl-O—C(═O)(C3-C7-cycloalkyl)NH₂, C1-C2-alkyl-O—C(═O)(C1-C6-alkyl)NH₂, aryl and heteroaryl, wherein aryl or heteroaryl are optionally substituted with 1, 2, or 3 groups each independently selected from halo and C1-C6-alkyl
R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halo, carboxy, OCF₃, OCHF₂ and

In one embodiment subject matter of the present invention is a compound according to Formula I in which R1 is phenyl or pyridyl, optionally substituted once, twice, or thrice by halo, C1-C4-alkyl, C3-C6-cycloalkyl, C1-C4-haloalkyl or

In one embodiment subject matter of the present invention is a compound according to Formula I in which R2 is selected from the group comprising H and methyl.

In one embodiment subject matter of the present invention is a compound according to Formula I in which R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, C2-C6-alkyl-O-C1-C6-alkyl, optionally substituted with 1, 2, or 3 groups each independently selected from OH, halo, carboxy, C3-C7-heterocyclo alkyl , C1-C6-alkyl, C1-C6-haloalkyl, C1-C6-hydroxyalkyl, C1-C6-alkyl-O-C1-C6-alkyl, C1-C6-alkyl-O-C1-C6-halo alkyl, C1-C6-alkyl-S-C1-C6-alkyl, C1-C6-alkyl-SO₂-C1-C6-alkyl, C1-C6-alkyl-C≡N, C1-C2-alkyl-O-C3-C6-cycloalkyl, C1-C2-alkyl-C3-C7-heterocyclo alkyl, C1-C2-alkyl-O—C(═O)(C3-C7-cycloalkyl)NH₂, C1-C2-alkyl-O—C(═O)(C1-C6-alkyl)NH₂, aryl and heteroaryl, wherein aryl or heteroaryl are optionally substituted with 1, 2, or 3 groups each independently selected from halo and C1-C6-alkyl

In one embodiment subject matter of the present invention is a compound according to Formula I in which R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halo, carboxy, OCF₃, OCHF₂ and C≡N.

One embodiment of the invention is a compound of Formula I or a pharmaceutically acceptable salt thereof according to the invention, for use in the prevention or treatment of an HBV infection in subject.

One embodiment of the invention is a pharmaceutical composition comprising a compound of Formula I or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula I or a pharmaceutically acceptable salt thereof according to the present invention.

A further embodiment of the invention is a compound of Formula II or a pharmaceutically acceptable salt thereof according to the invention, for use in the prevention or treatment of an HBV infection in subject in need thereof.

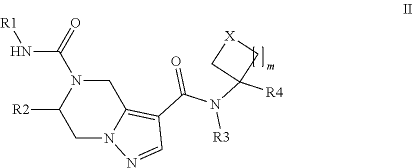

in which
R1 is phenyl or pyridyl,optionally substituted once, twice, or thrice by halo, C1-C4-alkyl, C3-C6-cycloalkyl, C1-C4-haloalkyl or C≡N
R2 is H or methyl
R3 is C1-C4 alkyl said C1-C4-alkyl is unsubstituted or substituted once, twice, or thrice with deuterium, OH or halo
R4 is selected from the group comprising C1-C2-alkyl-O-C1-C4-alkyl, C1-C2-hydroxyalkyl, C1-C2-alkyl-O-C1-C4-halo alkyl, C1-C2-alkyl-NH-C1-C4-halo alkyl, C1-C2-alkyl-O-C3-C6-cycloalkyl, C1-C2-alkyl-S-C1-C4-alkyl, C1-C2-alkyl-SO₂-C1-C4-alkyl, C1-C2-alkyl-CN, C1-C2-alkyl-C3-C7-heterocyclo alkyl, C1-C2-alkyl-O—C(═O)(C3-C7-cycloalkyl)NH₂, C1-C2-alkyl-O—C(═O)(C1-C6-alkyl)NH₂, aryl and heteroaryl, wherein aryl or heteroaryl are optionally substituted once, twice or thrice with halo or C1-C6 alkyl
R3 and R4 are optionally connected to form a five, six or seven membered heterocyclic ring, wherein said heterocyclic ring is unsubstituted or substituted once, twice or thrice with halo, OH, carboxy, OCF₃, OCHF₂ or C≡N
X is O, CH₂, or NR5
m is 0, 1, 2 or 3
R5 is H or C1-C4-alkyl.

In one embodiment of the invention subject matter of the present invention is a compound of Formula II in which R1 is phenyl or pyridyl, optionally substituted once, twice, or thrice by halo, C1-C4-alkyl, C3-C6-cycloalkyl, C1-C4-haloalkyl or C≡N.

In one embodiment of the invention subject matter of the invention is a compound of Formula II in which R2 is H or methyl.

In one embodiment of the invention subject matter of the invention is a compound of Formula II in which R3 is C1-C4 alkyl said C1-C4-alkyl is unsubstituted or substituted once, twice, or thrice with halo or OH.

In one embodiment of the invention subject matter of the invention is a compound of Formula II in which R4 is C1-C2-alkyl-O-C1-C4-alkyl, C1-C2-hydroxyalkyl, C1-C2-alkyl-O-C1-C4-haloalkyl, C1-C2-alkyl-NH-C1-C4-haloalkyl, C1-C2-alkyl-O-C3-C6-cycloalkyl, C1-C2-alkyl-S-C1-C4-alkyl, C1-C2-alkyl-SO₂-C1-C4-alkyl, C1-C2-alkyl-C≡N, C1-C2-alkyl-C3-C7-heterocycloalkyl, C1-C2-alkyl-O—C(═O)(C3-C7-cycloalkyl)NH₂, C1-C2-alkyl-O—C(═O)(C1-C6-alkyl)NH₂, aryl or heteroaryl, wherein aryl or heteroaryl are optionally substituted once, twice or thrice with halo or C1-C6 alkyl.

In one embodiment of the invention subject matter of the invention is a compound of Formula II in which X is O, CH₂, or NR5.

In one embodiment of the invention subject matter of the invention is a compound of Formula II in which R5 is H or C1-C4-alkyl.

In one embodiment of the invention subject matter of the invention is a compound of Formula II in which m is 0, 1, 2 or 3

In one embodiment of the invention subject matter of the invention is a compound of Formula II in which R3 and R4 are optionally connected to form a five, six or seven membered carbocylic or heterocyclic ring, said carbocyclic or heterocyclic ring is unsubstituted or substituted once, twice or thrice with halo, carboxy, OH, OCF₃, OCHF₂ or C≡N.

One embodiment of the invention is a compound of Formula II or a pharmaceutically acceptable salt thereof according to the invention, for use in the prevention or treatment of an HBV infection in subject.

One embodiment of the invention is a pharmaceutical composition comprising a compound of Formula II or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula II or a pharmaceutically acceptable salt thereof according to the present invention.

A further embodiment of the invention is a compound of Formula II or a pharmaceutically acceptable salt thereof according to the invention, for use in the prevention or treatment of an HBV infection in subject in need thereof

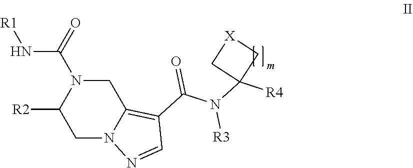

in which
R1 is phenyl or pyridyl,optionally substituted once, twice, or thrice by halo, C1-C4-alkyl, C3-C6-cycloalkyl, C1-C4-haloalkyl or C≡N
R2 is H or methyl
R3 is C1-C4 alkyl said C1-C4-alkyl is unsubstituted or substituted once, twice, or thrice with deuterium or halo
R4 is selected from the group comprising C1-C2-alkyl-O-C1-C4-alkyl, C1-C2-hydroxyalkyl, C1-C2-alkyl-O-C1-C4-haloalkyl, C1-C2-alkyl-O-C3-C6-cycloalkyl , C1-C2-alkyl-S-C1-C4-alkyl, C1-C2-alkyl-SO₂-C1-C4-alkyl, C1-C2-alkyl-C≡N, C1-C2-alkyl-C3-C7-heterocycloalkyl, C1-C2-alkyl-O—C(═O)(C3-C7-cycloalkyl)NH₂, C1-C2-alkyl-O—C(═O)(C1-C6-alkyl)NH₂, aryl and heteroaryl, wherein aryl or heteroaryl are optionally substituted once, twice or thrice with halo or C1-C6 alkyl
R3 and R4 are optionally connected to form a five, six or seven membered heterocyclic ring, wherein said heterocyclic ring is unsubstituted or substituted once, twice or thrice with halo, OH, carboxy, OCF₃, OCHF₂ or C≡N
X is O, CH₂, or NR5
m is 0, 1 or 2
R5 is H or C1-C4-alkyl.

In one embodiment of the invention subject matter of the present invention is a compound of Formula II in which R1 is phenyl or pyridyl, optionally substituted once, twice, or thrice by halo, C1-C4-alkyl, C3-C6-cycloalkyl, C1-C4-haloalkyl or

In one embodiment of the invention subject matter of the invention is a compound of Formula II in which R2 is H or methyl.

In one embodiment of the invention subject matter of the invention is a compound of Formula II in which R3 is C1-C4 alkyl said C1-C4-alkyl is unsubstituted or substituted once, twice, or thrice with halo.

In one embodiment of the invention subject matter of the invention is a compound of Formula II in which R4 is C1-C2-alkyl-O-C1-C4-alkyl, C1-C2-hydroxyalkyl, C1-C2-alkyl-O-C1-C4-haloalkyl, C1-C2-alkyl-O-C3-C6-cycloalkyl, C1-C2-alkyl-S-C1-C4-alkyl, C1-C2-alkyl-SO₂-C1-C4-alkyl, C1-C2-alkyl-C≡N, C1-C2-alkyl-C3-C7-heterocycloalkyl, C1-C2-alkyl-O—C(═O)(C3-C7-cycloalkyl)NH₂, C1-C2-alkyl-O—C(═O)(C1-C6-alkyl)NH₂, aryl or heteroaryl, wherein aryl or heteroaryl are optionally substituted once, twice or thrice with halo or C1-C6 alkyl.

In one embodiment of the invention subject matter of the invention is a compound of Formula II in which X is O, CH₂, or NR5.

In one embodiment of the invention subject matter of the invention is a compound of Formula II in which R5 is H or C1-C4-alkyl.

In one embodiment of the invention subject matter of the invention is a compound of Formula II in which m is 0, 1 or 2.

In one embodiment of the invention subject matter of the invention is a compound of Formula II in which R3 and R4 are optionally connected to form a five, six or seven membered carbocylic or heterocyclic ring, said carbocyclic or heterocyclic ring is unsubstituted or substituted once, twice or thrice with halo, carboxy, OH, OCF₃, OCHF₂ or C≡N.

One embodiment of the invention is a compound of Formula II or a pharmaceutically acceptable salt thereof according to the invention, for use in the prevention or treatment of an HBV infection in subject.

One embodiment of the invention is a pharmaceutical composition comprising a compound of Formula II or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula II or a pharmaceutically acceptable salt thereof according to the present invention.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof. All before mentioned doses refer to daily doses per patient.

In general it is contemplated that an antiviral effective daily amount would be from about 0.01 to about 50 mg/kg, or about 0.01 to about 30 mg/kg body weight. It maybe appropriate to administer the required dose as two, three, four or more sub-doses at appropriate intervals throughout the day. Said sub-doses may be formulated as unit dosage forms, for example containing about 1 to about 500 mg, or about 1 to about 300 mg or about 1 to about 100 mg, or about 2 to about 50 mg of active ingredient per unit dosage form.

The compounds of the invention may, depending on their structure, exist as salts, solvates or hydrates. The invention therefore also encompasses the salts, solvates or hydrates and respective mixtures thereof.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

### DEFINITIONS

Listed below are definitions of various terms used to describe this invention. These definitions apply to the terms as they are used throughout this specification and claims unless otherwise limited in specific instances either individually or as part of a larger group.

Unless defined otherwise all technical and scientific terms used herein generally have the same meaning as commonly understood by one of ordinary skill in the art to which this invention belongs. Generally the nomenclature used herein and the laboratory procedures in cell culture, molecular genetics, organic chemistry and peptide chemistry are those well known and commonly employed in the art.

As used herein the articles “a” and “an” refer to one or to more than one (i.e. to at least one) of the grammatical object of the article. By way of example, “an element” means one element or more than one element. Furthermore, use of the term “including” as well as other forms such as “include”, “includes” and “included”, is not limiting.

As used herein the term “capsid assembly modulator” refers to a compound that disrupts or accelerates or inhibits or hinders or delays or reduces or modifies normal capsid assembly (e.g. during maturation) or normal capsid disassembly (e.g. during infectivity) or perturbs capsid stability, thereby inducing aberrant capsid morphology or aberrant capsid function. In one embodiment, a capsid assembly modulator accelerates capsid assembly or disassembly thereby inducing aberrant capsid morphology. In another embodiment a capsid assembly modulator interacts (e.g. binds at an active site, binds at an allosteric site or modifies and/or hinders folding and the like), with the major capsid assembly protein (HBV-CP), thereby disrupting capsid assembly or disassembly. In yet another embodiment a capsid assembly modulator causes a perturbation in the structure or function of HBV-CP (e.g. the ability of HBV-CP to assemble, disassemble, bind to a substrate, fold into a suitable conformation or the like which attenuates viral infectivity and/or is lethal to the virus).

As used herein the term “treatment” or “treating” is defined as the application or administration of a therapeutic agent i.e., a compound of the invention (alone or in combination with another pharmaceutical agent) to a patient, or application or administration of a therapeutic agent to an isolated tissue or cell line from a patient (e.g. for diagnosis or ex vivo applications) who has an HBV infection, a symptom of HBV infection, or the potential to develop an HBV infection with the purpose to cure, heal, alleviate, relieve, alter, remedy, ameliorate, improve or affect the HBV infection, the symptoms of HBV infection or the potential to develop an HBV infection. Such treatments may be specifically tailored or modified based on knowledge obtained from the field of pharmacogenomics.

As used herein the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein the term “patient”, “individual” or “subject” refers to a human or a non-human mammal. Non-human mammals include for example livestock and pets such as ovine, bovine, porcine, feline, and murine mammals. Preferably the patient, subject, or individual is human.

As used herein the terms “effective amount”, “pharmaceutically effective amount”, and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein the term “pharmaceutically acceptable” refers to a material such as a carrier or diluent which does not abrogate the biological activity or properties of the compound and is relatively non-toxic i.e. the material may be administered to an individual without causing undesirable biological effects or interacting in a deleterious manner with any of the components of the composition in which it is contained.

As used herein the term “pharmaceutically acceptable salt” refers to derivatives of the disclosed compounds wherein the parent compound is modified by converting an existing acid or base moiety to its salt form. Examples of pharmaceutically acceptable salts include but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent or in a mixture of the two; generally nonaqueous media like ether, ethyl acetate, ethanol, isopropanol, or acetonitrile are preferred. Lists of suitable salts are found in Remington's Pharmaceutical Sciences 17^{th} ed. Mack Publishing Company, Easton, Pa., 1985 p. 1418 and Journal of Pharmaceutical Science, 66, 2 (1977), each of which is incorporated herein by reference in its entirety.

As used herein the term “composition” or “pharmaceutical composition” refers to a mixture of at least one compound useful within the invention with a pharmaceutically acceptable carrier. The pharmaceutical composition facilitates administration of the compound to a patient or subject.

Multiple techniques of administering a compound exist in the art including but not limited to intravenous, oral, aerosol, rectal, parenteral, ophthalmic, pulmonary and topical administration.

As used herein the term “pharmaceutically acceptable carrier” means a pharmaceutically acceptable material, composition or carrier such as a liquid or solid filler, stabilizer, dispersing agent, suspending agent, diluent, excipient, thickening agent, solvent or encapsulating material involved in carrying or transporting a compound useful within the invention within or to the patient such that it may perform its intended function. Typically such constructs are carried or transported from one organ, or portion of the body, to another organ or portion of the body. Each carrier must be “acceptable” in the sense of being compatible with the other ingredients of the formulation including the compound use within the invention and not injurious to the patient. Some examples of materials that may serve as pharmaceutically acceptable carriers include: sugars, such as lactose, glucose and sucrose; starches such as corn starch and potato starch; cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols such as propylene glycol; polyols such as glycerin, sorbitol, mannitol and polyethylene glycol; esters such as ethyl oleate and ethyl laurate; agar; buffering agents, such as magnesium hydroxide and aluminium hydroxide; surface active agents; alginic acid; pyrogen-free water; isotonic saline; Ringer's solution; ethyl alcohol; phosphate buffer solutions and other non-toxic compatible substances employed in pharmaceutical formulations.

As used herein “pharmaceutically acceptable carrier” also includes any and all coatings, antibacterial and antifungal agents and absorption delaying agents and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described for example in Remington's Pharmaceutical Sciences (Genaro, Ed., Mack Publishing Company, Easton, Pa., 1985) which is incorporated herein by reference.

As used herein, the term “substituted” means that an atom or group of atoms has replaced hydrogen as the substituent attached to another group.

As used herein, the term “comprising” also encompasses the option “consisting of”.

As used herein, the term “alkyl” by itself or as part of another substituent means, unless otherwise stated, a straight or branched chain hydrocarbon having the number of carbon atoms designated (i.e. C1-C6-alkyl means one to six carbon atoms) and includes straight and branched chains. Examples include methyl, ethyl, propyl, isopropyl, butyl, isobutyl, tert-butyl, pentyl, neopentyl, and hexyl. In addition, the term “alkyl” by itself or as part of another substituent can also mean a C1-C3 straight chain hydrocarbon substituted with a C3-C5-carbocylic ring. Examples include (cyclopropyl)methyl, (cyclobutyl)methyl and (cyclopentyl)methyl. For the avoidance of doubt, where two alkyl moieties are present in a group, the alkyl moieties may be the same or different.

As used herein the term “alkenyl” denotes a monovalent group derived from a hydrocarbon moiety containing at least two carbon atoms and at least one carbon-carbon double bond of either E or Z stereochemistry. The double bond may or may not be the point of attachment to another group. Alkenyl groups (e.g. C2-C8-alkenyl) include, but are not limited to for example ethenyl, propenyl, prop-1-en-2-yl, butenyl, methyl-2-buten-1-yl, heptenyl and octenyl. For the avoidance of doubt, where two alkenyl moieties are present in a group, the alkyl moieties may be the same or different.

As used herein, a C2-C6-alkynyl group or moiety is a linear or branched alkynyl group or moiety containing from 2 to 6 carbon atoms, for example a C2-C4 alkynyl group or moiety containing from 2 to 4 carbon atoms. Exemplary alkynyl groups include —C≡CH or —CH₂≡CC, as well as 1- and 2-butynyl, 2-pentynyl, 3-pentynyl, 4-pentynyl, 2-hexynyl, 3-hexynyl, 4-hexynyl and 5-hexynyl. For the avoidance of doubt, where two alkynyl moieties are present in a group, they may be the same or different.

As used herein, the term “halo” or “halogen” alone or as part of another substituent means unless otherwise stated a fluorine, chlorine, bromine, or iodine atom, preferably fluorine, chlorine, or bromine, more preferably fluorine or chlorine. For the avoidance of doubt, where two halo moieties are present in a group, they may be the same or different.

As used herein, a C1-C6-alkoxy group or C2-C6-alkenyloxy group is typically a said C1-C6-alkyl (e.g. a C1-C4 alkyl) group or a said C2-C6-alkenyl (e.g. a C2-4 alkenyl) group respectively which is attached to an oxygen atom.

As used herein the term “aryl” employed alone or in combination with other terms, means unless otherwise stated a carbocyclic aromatic system containing one or more rings (typically one, two or three rings) wherein such rings may be attached together in a pendant manner such as a biphenyl, or may be fused, such as naphthalene. Examples of aryl groups include phenyl, anthracyl, and naphthyl. Preferred examples are phenyl (e.g. C6-aryl) and biphenyl (e.g. C12-aryl). In some embodiments aryl groups have from six to sixteen carbon atoms. In some embodiments aryl groups have from six to twelve carbon atoms (e.g. C6-C12-aryl). In some embodiments, aryl groups have six carbon atoms (e.g. C6-aryl).

As used herein the terms “heteroaryl” and “heteroaromatic” refer to a heterocycle having aromatic character containing one or more rings (typically one, two or three rings). Heteroaryl substituents may be defined by the number of carbon atoms e.g. C1-C9-heteroaryl indicates the number of carbon atoms contained in the heteroaryl group without including the number of heteroatoms. For example a C1-C9-heteroaryl will include an additional one to four heteroatoms. A polycyclic heteroaryl may include one or more rings that are partially saturated. Non-limiting examples of heteroaryls include:

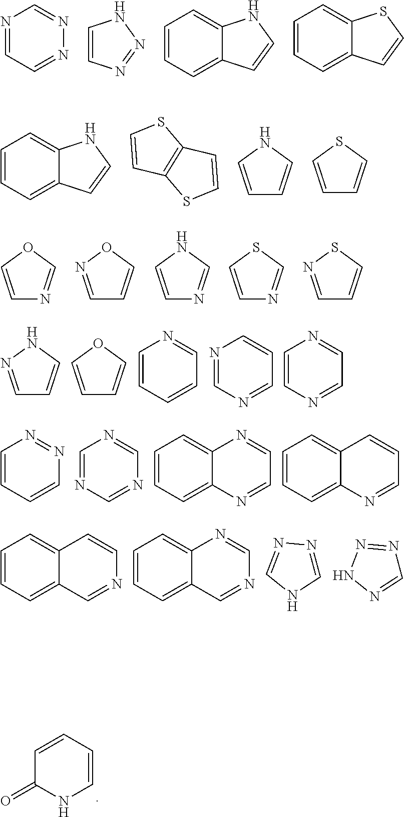

Additional non-limiting examples of heteroaryl groups include pyridyl, pyrazinyl, pyrimidinyl (including e.g. 2-and 4-pyrimidinyl), pyridazinyl, thienyl, furyl, pyrrolyl (including e.g., 2-pyrrolyl), imidazolyl, thiazolyl, oxazolyl, pyrazolyl (including e.g. 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Non-limiting examples of polycyclic heterocycles and heteroaryls include indolyl (including 3-, 4-, 5-, 6-and 7-indolyl), indolinyl, quinolyl, tetrahydroquinolyl, isoquinolyl (including, e.g. 1-and 5-isoquinolyl), 1,2,3,4-tetrahydroisoquinolyl, cinnolinyl, quinoxalinyl (including, e .g 2-and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e .g. 3-, 4-, 5-, 6-, and 7-benzofuryl), 2,3-dihydrobenzofuryl, 1,2-benzisoxazolyl, benzothienyl (including e.g. 3-, 4-, 5-, 6-, and 7-benzothienyl), benzoxazolyl, benzothiazolyl (including e.g. 2-benzothiazolyl and 5-benzothiazolyl), purinyl, benzimidazolyl (including e.g., 2-benzimidazolyl), benzotriazolyl, thioxanthinyl, carbazolyl, carbolinyl, acridinyl, pyrrolizidinyl and quinolizidinyl.

As used herein the term “haloalkyl” is typically a said alkyl, alkenyl, alkoxy or alkenoxy group respectively wherein any one or more of the carbon atoms is substituted with one or more said halo atoms as defined above. Haloalkyl embraces monohaloalkyl, dihaloalkyl, and polyhaloalkyl radicals. The term “haloalkyl”includes but is not limited to fluoromethyl, 1-fluoroethyl, difluoromethyl, 2,2-difluoroethyl, 2,2,2-trifluoroethyl, trifluoromethyl, chloromethyl, dichloromethyl, trichloromethyl, pentafluoroethyl, difluoromethoxy, and trifluoromethoxy.

As used herein, a C1-C6-hydroxyalkyl group is a said C1-C6 alkyl group substituted by one or more hydroxy groups. Typically, it is substituted by one, two or three hydroxyl groups. Preferably, it is substituted by a single hydroxy group.

As used herein, a C1-C6-aminoalkyl group is a said C1-C6 alkyl group substituted by one or more amino groups. Typically, it is substituted by one, two or three amino groups. Preferably, it is substituted by a single amino group.

As used herein, a C1-C4-carboxyalkyl group is a said C1-C4 alkyl group substituted by carboxyl group.

As used herein, a C1-C4-carboxamidoalkyl group is a said C1-C4 alkyl group substituted by a substituted or unsubstituted carboxamide group.

As used herein, a C1-C4-acylsulfonamido-alkyl group is a said C1-C4 alkyl group substituted by an acylsulfonamide group of general formula C(═O)NHSO₂CH₃ or C(═O)NHSO₂-c-Pr.

As used herein the term “cycloalkyl” refers to a monocyclic or polycyclic nonaromatic group wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having 3 to 10 ring atoms (C3-C10-cycloalkyl), groups having 3 to 8 ring atoms (C3-C8-cycloalkyl), groups having 3 to 7 ring atoms (C3-C7-cycloalkyl) and groups having 3 to 6 ring atoms (C3-C6-cycloalkyl). Illustrative examples of cycloalkyl groups include, but are not limited to the following moieties:

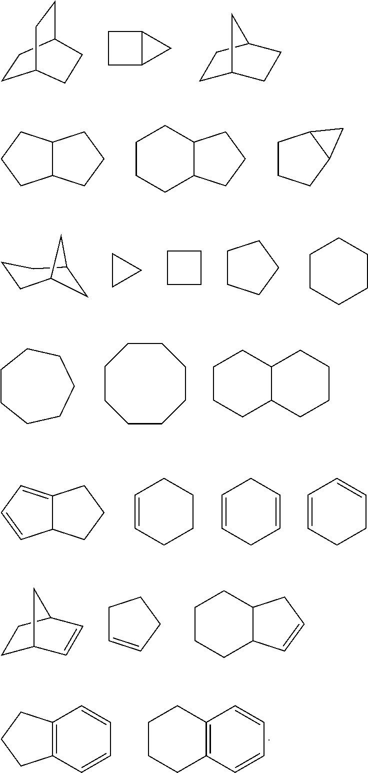

Monocyclic cycloalkyls include but are not limited to cyclopropyl, cyclobutyl, cyclopentyl, cyclohexyl, cycloheptyl, and cyclooctyl. Dicyclic cycloalkyls include but are not limited to tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups both of which refer to a nonaromatic carbocycle as defined herein which contains at least one carbon-carbon double bond or one carbon-carbon triple bond.

As used herein, the term “spirocyclic” refers to any compound containing two or more rings wherein two of the rings have one ring carbon in common.

As used herein the terms “heterocycloalkyl” and “heterocyclyl” refer to a heteroalicyclic group containing one or more rings (typically one, two or three rings), that contains one to four ring heteroatoms each selected from oxygen, sulfur and nitrogen. In one embodiment each heterocyclyl group has from 3 to 10 atoms in its ring system with the proviso that the ring of said group does not contain two adjacent oxygen or sulfur atoms. In one embodiment each heterocyclyl group has a fused bicyclic ring system with 3 to 10 atoms in the ring system, again with the proviso that the ring of said group does not contain two adjacent oxygen or sulfur atoms. In one embodiment each heterocyclyl group has a bridged bicyclic ring system with 3 to 10 atoms in the ring system, again with the proviso that the ring of said group does not contain two adjacent oxygen or sulfur atoms. In one embodiment each heterocyclyl group has a spiro-bicyclic ring system with 3 to 10 atoms in the ring system, again with the proviso that the ring of said group does not contain two adjacent oxygen or sulfur atoms. Heterocyclyl substituents may be alternatively defined by the number of carbon atoms e.g. C2-C8-heterocyclyl indicates the number of carbon atoms contained in the heterocyclic group without including the number of heteroatoms. For example a C2-C8-heterocyclyl will include an additional one to four heteroatoms. In another embodiment the heterocycloalkyl group is fused with an aromatic ring. . In another embodiment the heterocycloalkyl group is fused with a heteroaryl ring. In one embodiment the nitrogen and sulfur heteroatoms may be optionally oxidized and the nitrogen atom may be optionally quaternized. The heterocyclic system may be attached, unless otherwise stated, at any heteroatom or carbon atom that affords a stable structure. An example of a 3-membered heterocyclyl group includes and is not limited to aziridine. Examples of 4-membered heterocycloalkyl groups include, and are not limited to azetidine and a beta-lactam. Examples of 5-membered heterocyclyl groups include, and are not limited to pyrrolidine, oxazolidine and thiazolidinedione. Examples of 6-membered heterocycloalkyl groups include, and are not limited to, piperidine, morpholine, piperazine, N-acetylpiperazine and N-acetylmorpholine. Other non-limiting examples of heterocyclyl groups are

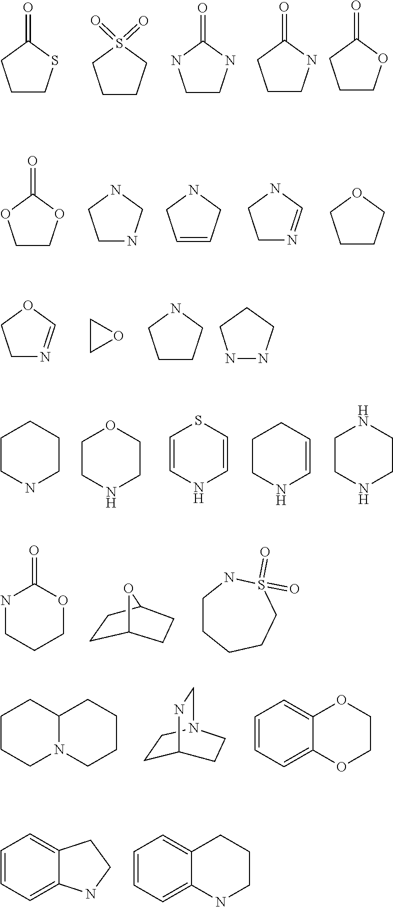

Examples of heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, 1,3-dioxolane, homopiperazine, homopiperidine, 1,3-dioxepane, 47-dihydro-1,3-dioxepin, and hexamethyleneoxide. The terms “C3-C7-heterocycloalkyl” includes but is not limited to tetrahydrofuran-2-yl, tetrahydrofuran-3-yl, 3-oxabicyclo [3.1.0]hexan-6-yl, 3-azabicyclo [3.1.0]hexan- 6-yl, tetrahydropyran-4-yl, tetrahydropyran-3-yl, tetrahydropyran-2-yl, and azetidin-3-yl.

As used herein, the term “aromatic” refers to a carbocycle or heterocycle with one or more polyunsaturated rings and having aromatic character i.e. having (4n+2) delocalized π(pi) electrons where n is an integer.

As used herein, the term “acyl”, employed alone or in combination with other terms, means, unless otherwise stated, to mean to an alkyl, cycloalkyl, heterocycloalkyl, aryl or heteroaryl group linked via a carbonyl group.

As used herein, the terms “carbamoyl” and “substituted carbamoyl”, employed alone or in combination with other terms, means, unless otherwise stated, to mean a carbonyl group linked to an amino group optionally mono or di-substituted by hydrogen, alkyl, cycloalkyl, heterocycloalkyl, aryl or heteroaryl. In some embodiments, the nitrogen substituents will be connected to form a heterocyclyl ring as defined above.

As used herein, the term “carboxy” and by itself or as part of another substituent means, unless otherwise stated, a group of formula C(═O)OH.

As used herein, the term “carboxyl ester” by itself or as part of another substituent means, unless otherwise stated, a group of formula C(═O)OX, wherein X is selected from the group consisting of C1-C6-alkyl, C3-C7-cycloalkyl, and aryl.

As used herein the term “prodrug” represents a derivative of a compound of Formula I or Formula II which is administered in a form which, once administered, is metabolised in vivo into an active metabolite also of Formula I or Formula II.

Various forms of prodrug are known in the art. For examples of such prodrugs see: Design of Prodrugs, edited by H. Bundgaard, (Elsevier, 1985) and Methods in Enzymology, Vol. 42, p. 309-396, edited by K. Widder, et al. (Academic Press, 1985); A Textbook of Drug Design and Development, edited by Krogsgaard-Larsen and H. Bundgaard, Chapter 5 “Design and Application of Prodrugs” by H. Bundgaard p. 113-191 (1991); H. Bundgaard, Advanced Drug Delivery Reviews 8, 1-38 (1992); H. Bundgaard, et al., Journal of Pharmaceutical Sciences, 77, 285 (1988); and N. Kakeya, et al., Chem. Pharm. Bull., 32, 692 (1984).

Examples of prodrugs include cleavable esters of compounds of Formula I or Formula II. An in vivo cleavable ester of a compound of the invention containing a carboxy group is, for example, a pharmaceutically acceptable ester which is cleaved in the human or animal body to produce the parent acid. Suitable pharmaceutically acceptable esters for carboxy include C1-C6 alkyl ester, for example methyl or ethyl esters; C1-C6 alkoxymethyl esters, for example methoxymethyl ester; C1-C6 acyloxymethyl esters; phthalidyl esters; C3-C8 cycloalkoxycarbonyloxy C1-C6 alkyl esters, for example 1-cyclohexylcarbonyloxyethyl; 1-3-dioxolan-2-ylmethylesters, for example 5-methyl-1,3-dioxolan-2-ylmethyl; C1-C6 alkoxycarbonyloxyethyl esters, for example 1-methoxycarbonyloxyethyl; aminocarbonylmethyl esters and mono-or di-N-(C1-C6 alkyl) versions thereof, for example N, N-dimethylaminocarbonylmethyl esters and N-ethylaminocarbonylmethyl esters; and may be formed at any carboxy group in the compounds of the invention.

An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include C1-C6-acyl esters, for example acetyl esters; and benzoyl esters wherein the phenyl group may be substituted with aminomethyl or N-substituted mono-or di-C1-C6 alkyl aminomethyl, for example 4-aminomethylbenzoyl esters and 4-N,N-dimethylaminomethylbenzoyl esters.

Preferred prodrugs of the invention include acetyloxy and carbonate derivatives. For example, a hydroxy group of a compound of Formula I or Formula II can be present in a prodrug as —O—CORⁱ or —O—C(O)ORⁱ where Rⁱ is unsubstituted or substituted C1-C4 alkyl. Substituents on the alkyl groups are as defined earlier. Preferably the alkyl groups in R′ is unsubstituted, preferable methyl, ethyl, isopropyl or cyclopropyl.

Other preferred prodrugs of the invention include amino acid derivatives. Suitable amino acids include α-amino acids linked to compounds of Formula I or Formula II via their C(O)OH group. Such prodrugs cleave in vivo to produce compounds of Formula I or Formula II bearing a hydroxy group. Accordingly, such amino acid groups are preferably employed positions of Formula I or Formula II where a hydroxy group is eventually required. Exemplary prodrugs of this embodiment of the invention are therefore compounds of Formula I or Formula II bearing a group of Formula —OC(O)—CH(NH₂)Rⁱⁱ where Rⁱⁱ is an amino acid side chain. Preferred amino acids include glycine, alanine, valine and serine. The amino acid can also be functionalised, for example the amino group can be alkylated. A suitable functionalised amino acid is N,N-dimethylglycine. Preferably the amino acid is valine.

Other preferred prodrugs of the invention include phosphoramidate derivatives. Various forms of phosphoramidate prodrugs are known in the art. For example of such prodrugs see Serpi et al., Curr. Protoc. Nucleic Acid Chem. 2013, Chapter 15, Unit 15.5 and Mehellou et al., ChemMedChem, 2009, 4 pp. 1779-1791. Suitable phosphoramidates include (phenoxy)-α-amino acids linked to compounds of Formula I or Formula II via their -OH group. Such prodrugs cleave in vivo to produce compounds of Formula I or Formula II bearing a hydroxy group. Accordingly, such phosphoramidate groups are preferably employed positions of Formula I or Formula II where a hydroxy group is eventually required. Exemplary prodrugs of this embodiment of the invention are therefore compounds of Formula I or Formula II bearing a group of Formula —OP(O)(ORⁱⁱⁱ)R^{iv} where Rⁱⁱⁱ is alkyl, cycloalkyl, aryl or heteroaryl, and R^{iv} is a group of Formula —NH—CH(R^{v})C(O)OR^{vi}, wherein R^{v} is an amino acid side chain and R^{vi} is alkyl, cycloalkyl, aryl or heterocyclyl. Preferred amino acids include glycine, alanine, valine and serine. Preferably the amino acid is alanine. R^{v} is preferably alkyl, most preferably isopropyl.

Subject matter of the present invention is also a method of preparing the compounds of the present invention. Subject matter of the invention is, thus, a method for the preparation of a compound of Formula I according to the present invention by reacting a compound of Formula III

R1—N═C═O III

in which R1 is as above-defined, with a compound of Formula IV

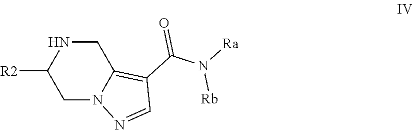

in which R2, R^{a} and R^{b} are as above-defined.

### EXAMPLES

The invention is now described with reference to the following Examples. These Examples are provided for the purpose of illustration only, and the invention is not limited to these Examples, but rather encompasses all variations that are evident as a result of the teachings provided herein.

The HBV core protein modulators can be prepared in a number of ways. Schemes 1 and 2 illustrate the main routes employed for their preparation for the purpose of this application. To the chemist skilled in the art it will be apparent that there are other methodologies that will also achieve the preparation of these intermediates and Examples.

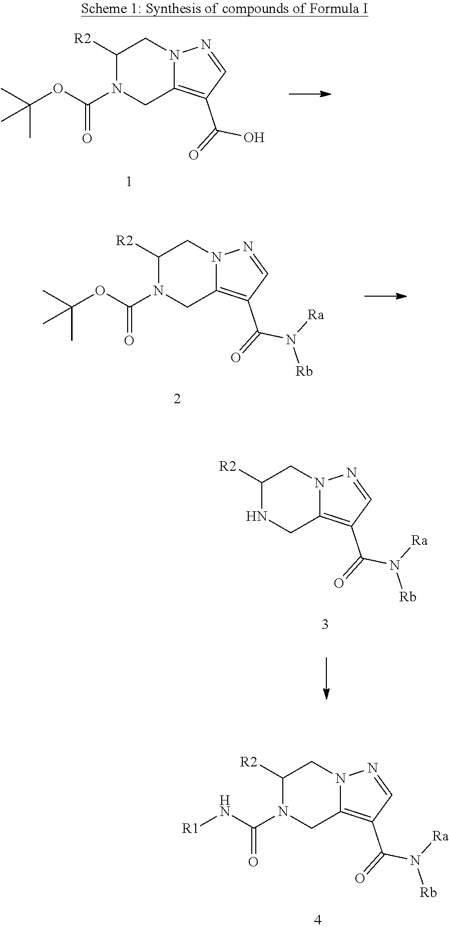

Compound 1 described in Scheme 1 is in step 1 coupled with an amine with methods known in literature (A. El-Faham, F. Albericio, Chem. Rev. 2011, 111, 6557-6602), e.g. with HATU to give a compound with the general structure 2. The nitrogen protective group of compound 2 in Scheme 1 is in step 2 deprotected (WO2018/011162, A. Isidro-Llobet et al., Chem. Rev., 2009, 109, 2455-2504), drawn as but not limited to Boc, e.g. with HCl to give an amine of general structure 3. Urea formation in step 3 with methods well known in literature (Pearson, A. J.; Roush, W. R.; Handbook of Reagents for Organic Synthesis, Activating Agents and Protecting Groups), e.g. with phenylisocyanate results in compounds of Formula I.

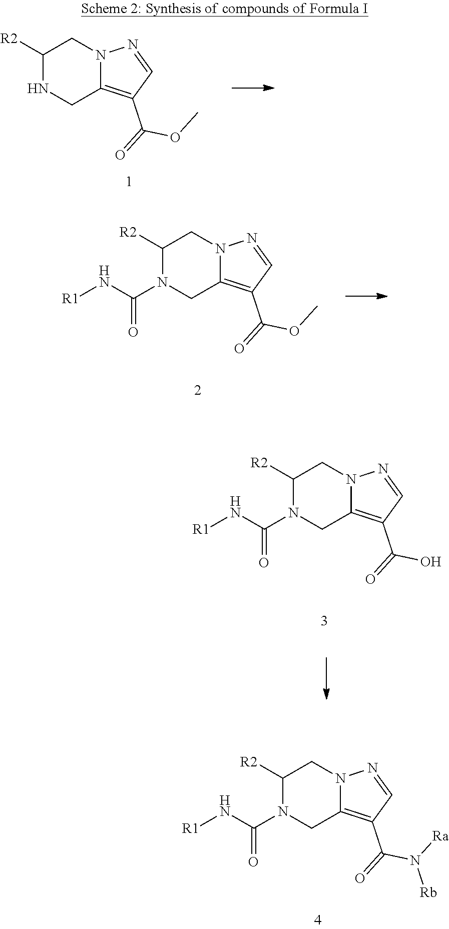

Compound 1 described in Scheme 2 is in step 1 transformed into the urea of general structure 2 with methods well known in literature (Pearson, A. J.; Roush, W. R.; Handbook of Reagents for Organic Synthesis, Activating Agents and Protecting Groups), e.g. with phenylisocyanate. The ester group of compound 2 is in step 2 hydrolyzed using methods known in the literature e.g. with LiOH (WO2015/0133428) to give a carboxylic acid of general structure 3. An amide coupling in step 3 with methods known in literature (A. El-Faham, F. Albericio, Chem. Rev. 2011, 111, 6557-6602), e.g. with HATU results in compounds of Formula I.

The following examples illustrate the preparation and properties of some specific compounds of the invention.

The following abbreviations are used:
A—DNA nucleobase adenine
ACN—acetonitrile
Ar—argon
BODIPY-FL—4,4-difluoro-5,7-dimethyl-4-bora-3a,4a-diaza-s-indacene-3-propionic acid (fluorescent dye)
Boc—tert-butoxycarbonyl
BnOH—benzyl alcohol
n-BuLi—n-butyl lithium
t-BuLi—t-butyl lithium
C—DNA nucleobase cytosine
CC₅₀—half-maximal cytotoxic concentration
CO₂ —carbon dioxide
CuCN—copper (I) cyanide
DCE—dichloroethane
DCM—dichloromethane
Dess-Martin periodinane—1,1,1-triacetoxy-1,1-dihydro-1,2-benziodoxol-3(1H)-one
DIPEA—diisopropylethylamine
DIPE—di-isopropyl ether
DMAP—4-dimethylaminopyridine
DMF—N,N-dimethylformamide
DMP—Dess-Martin periodinane
DMSO—dimethyl sulfoxide
DNA—deoxyribonucleic acid
DPPA—diphenylphosphoryl azide
DTT—dithiothreitol
EC₅₀—half-maximal effective concentration
EDCI—N-(3-dimethylaminopropyl)-N′-ethylcarbodiimide hydrochloride
Et₂O—diethyl ether
EtOAc—ethyl acetate
EtOH—ethanol
FL——five prime end labled with fluorescein
NEt₃—triethylamine
ELS—Evaporative Light Scattering
g—gram(s)
G—DNA nucleobase guanine
HBV—hepatitis B virus
HATU—2-(1H-7-azabenzotriazol-1-yl)-1,1,3,3-tetramethyl uronium hexafluorophosphate
HCl —hydrochloric acid
HEPES—4-(2-hydroxyethyl)-1-piperazineethanesulfonic acid
HOAt—1-hydroxy-7-azabenzotriazole
HOBt—1-hydroxybenzotriazole
HPLC—high performance liquid chromatography
IC₅₀—half-maximal inhibitory concentration
LC₆₄₀—3 prime end modification with fluorescent dye LightCycler® Red 640
LC/MS—liquid chromatography/mass spectrometry
LiAlH₄—lithium aluminium hydride
LiOH—lithium hydroxide
MeOH—methanol
MeCN—acetonitrile
MgSO₄—magnesium sulfate
mg—milligram(s)
min—minutes
mol—moles
mmol—millimole(s)
mL—millilitre(s)
MTBE—methyl tert-butyl ether
N₂—nitrogen
Na₂CO₃—sodium carbonate
NaHCO₃—sodium hydrogen carbonate
Na₂SO₄—sodium sulfate
NdeI—restriction enzyme recognizes CAATATG sites
NEt₃—triethylamine
NaH—sodium hydride
NaOH—sodium hydroxide
NH₃—ammonia
NH₄Cl—ammonium chloride
NMR—nuclear magnetic resonance
PAGE—polyacrylamide gel electrophoresis
PCR—polymerase chain reaction
qPCR—quantitative PCR
Pd/C—palladium on carbon
—PH—3 prime end phosphate modification
pTSA—4-toluene-sulfonic acid
Rt—retention time
r.t.—room temperature
sat.—saturated aqueous solution
SDS—sodium dodecyl sulfate
SI—selectivity index (═CC₅₀/EC₅₀)
STAB—sodium triacetoxyborohydride
T—DNA nucleobase thymine
TBAF—tetrabutylammonium fluoride
TFA—trifluoroacetic acid
THF—tetrahydrofuran
TLC—thin layer chromatography
Tris—tris(hydroxymethyl)-aminomethane
XhoI—restriction enzyme recognizes C\hat{ }TCGAG sites

### Compound Identification—NMR

For a number of compounds, NMR spectra were recorded using a Bruker DPX400 spectrometer equipped with a 5 mm reverse triple-resonance probe head operating at 400 MHz for the proton and 100 MHz for carbon. Deuterated solvents were chloroform-d (deuterated chloroform, CDCl₃) or d6-DMSO (deuterated DMSO, d6-dimethylsulfoxide). Chemical shifts are reported in parts per million (ppm) relative to tetramethylsilane (TMS) which was used as internal standard.

### Compound Identification—HPLC/MS

For a number of compounds, LC-MS spectra were recorded using the following analytical methods.

### Method A

Column—Reverse phase Waters Xselect CSH C₁₈ (50×2.1 mm, 3.5 micron)

Flow—0.8 mL/min, 25 degrees Celsius

Eluent A—95% acetonitrile+5% 10 mM ammonium carbonate in water (pH 9)

Eluent B—10 mM ammonium carbonate in water (pH 9)

Linear gradient t=0 min 5% A, t=3.5 min 98% A. t=6 min 98% A

### Method A2

Column—Reverse phase Waters Xselect CSH C₁₈ (50×2.1 mm, 3.5 micron)

Flow—0.8 mL/min, 25 degrees Celsius

Eluent A—95% acetonitrile +5% 10 mM ammonium carbonate in water (pH 9)

Eluent B—10 mM ammonium carbonate in water (pH 9)

Linear gradient t=0 min 5% A, t=4.5 min 98% A. t=6 min 98% A

### Method B

Column—Reverse phase Waters Xselect CSH C₁₈ (50×2.1 mm, 3.5 micron)

Flow—0.8 mL/min, 35 degrees Celsius

Eluent A—0.1% formic acid in acetonitrile

Eluent B—0.1% formic acid in water

Linear gradient t=0 min 5% A, t=3.5 min 98% A. t=6 min 98% A

### Method B2

Column—Reverse phase Waters Xselect CSH C₁₈ (50×2.1 mm, 3.5 micron)

Flow—0.8 mL/min, 40 degrees Celsius

Eluent A—0.1% formic acid in acetonitrile

Eluent B—0.1% formic acid in water

Linear gradient t=0 min 5% A, t=4.5 min 98% A. t=6 min 98% A

### Method C

Column—Reverse phase Waters Xselect CSH C₁₈ (50×2.1 mm, 3.5 micron)

Flow—1 mL/min, 35 degrees Celsius

Eluent A—0.1% formic acid in acetonitrile

Eluent B—0.1% formic acid in water

Linear gradient t=0 min 5% A, t=1.6 min 98% A. t=3 min 98% A

### Method D

Column—Phenomenex Gemini NX C₁₈ (50×2.0 mm, 3.0 micron)

Flow—0.8 mL/min, 35 degrees Celsius

Eluent A—95% acetonitrile+5% 10 mM ammoniumbicarbonate in water

Eluent B—10 mM ammoniumbicarbonate in water pH=9.0

Linear gradient t=0 min 5% A, t=3.5 min 98% A. t=6 min 98% A

### Method E

Column—Phenomenex Gemini NX C₁₈ (50×2.0 mm, 3.0 micron)

Flow—0.8 mL/min, 25 degrees Celsius

Eluent A—95% acetonitrile+5% 10 mM ammoniumbicarbonate in water

Eluent B—10 mM ammonium bicarbonate in water (pH 9)

Linear gradient t=0 min 5% A, t=3.5 min 30% A. t=7 min 98% A, t=10 min 98% A

### Method F

Column—Waters XSelect HSS C₁₈ (150×4.6 mm, 3.5 micron)

Flow—1.0 mL/min, 25 degrees Celsius

Eluent A—0.1% TFA in acetonitrile

Eluent B—0.1% TFA in water

Linear gradient t=0 min 2% A, t=1 min 2% A, t=15 min 60% A, t=20 min 60% A

### Method G

Column—Zorbax SB-C_{18 1.8} μm 4.6×15 mm Rapid Resolution cartridge (PN 821975-932)

Flow—3 mL/min

Eluent A—0.1% formic acid in acetonitrile

Eluent B—0.1% formic acid in water

Linear gradient t=0 min 0% A, t=1.8 min 100% A

### Method H

Column—Waters Xselect CSH C₁₈ (50×2.1 mm, 2.5 micron)

Flow—0.6 mL/min

Eluent A—0.1% formic acid in acetonitrile

Eluent B—0.1% formic acid in water

Linear gradient t=0 min 5% A, t=2.0 min 98% A, t=2.7 min 98% A

### Method J

Column—Reverse phase Waters Xselect CSH C₁₈ (50×2.1 mm, 2.5 micron)

Flow—0.6 mL/min

Eluent A—100% acetonitrile

Eluent B—10 mM ammonium bicarbonate in water (pH 7.9)

Linear gradient t=0 min 5% A, t=2.0 min 98% A, t=2.7 min 98% A

### Preparation of 6,6-difluoro-4-azaspiro[2.4]heptane

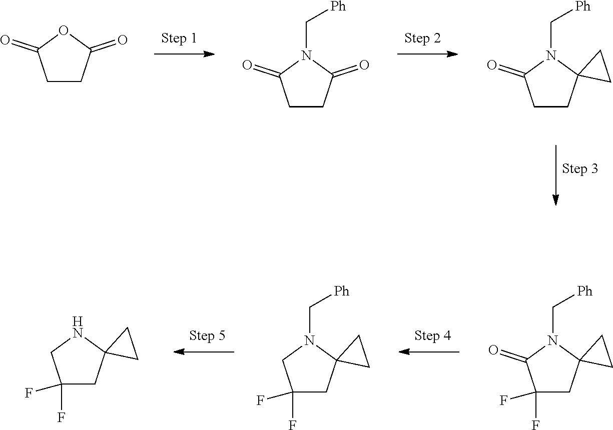 [SMILES: O=C1CCC(=O)O1 ; O=C1CCC(=O)N1Cc1ccccc1 ; O=C1CCC2(CC2)N1Cc1ccccc1 ; FC1(F)CNC2(CC2)C1 ; FC1(F)CN(Cc2ccccc2)C2(CC2)C1 ; O=C1N(Cc2ccccc2)C2(CC2)CC1(F)F]

Step 1: To a solution of succinic anhydride (100 g, 1000 mmol) in toluene (3000 mL) was added benzylamine (107 g, 1000 mmol). The solution was stirred at room temperature for 24 h, then heated at reflux with a Dean-Stark apparatus for 16 hours. The mixture was then concentrated under reduced pressure to give 1-benzylpyrrolidine-2,5-dione (170 g, 900 mmol, 90% yield).

Step 2: To a cooled (0° C.) mixture of 1-benzylpyrrolidine-2,5-dione (114 g, 600 mmol) and Ti(Oi-Pr)₄ (170.5 g, 600 mmol) in dry THF (2000 mL) under argon atmosphere was added dropwise a 3.4M solution of ethylmagnesium bromide in THF (1200 mmol). The mixture was warmed to room temperature and stirred for 4 h. BF₃.Et₂O (170 g, 1200 mmol) was then added dropwise and the solution stirred for 6 h. The mixture was cooled (0° C.) and 3N hydrochloric acid (500 mL) was added. The mixture was extracted twice with Et₂O, and the combined organic extracts washed with brine, dried and concentrated under reduced pressure to give 4-benzyl-4-azaspiro [2.4]heptan-5-one (30.2 g, 150 mmol, 25% yield).

Step 3: To a cooled (−78° C.) solution of 4-benzyl-4-azaspiro[2.4]heptan-5-one (34.2 g, 170 mmol) in dry THF (1000 mL) under argon was added LiHMDS in THF (1.1M solution, 240 mmol). The mixture was stirred for 1 h, then a solution of N-fluorobenzenesulfonimide (75.7 g, 240 mmol) in THF (200 mL) was added dropwise. The mixture was warmed to room temperature and stirred for 6 h. The mixture was then re-cooled (−78° C.) and LiHMDS added (1.1M solution in THF, 240 mmol).

The solution was stirred for 1 h, then N-fluorobenzenesulfonimide (75.7 g, 240 mmol) in THF (200 mL) was added dropwise. The mixture was warmed to room temperature and stirred for 6 h. The mixture was poured into a saturated solution of NH₄Cl (300 mL) and extracted twice with Et₂O. The combined organic extracts were washed with brine and concentrated under reduced pressure. Product was purified by column chromatography to provide 4-benzyl-6,6-difluoro-4-azaspiro[2.4]heptan-5-one (18 g, 75.9 mmol, 45% yield).

Step 4: To a warmed (40° C.) solution of BH₃.Me₂S (3.42 g, 45 mmol) in THF (200 mL) was added dropwise 4-benzyl-6,6-difluoro-4-azaspiro[2.4]heptan-5-one (11.9 g, 50 mmol). The mixture was stirred for 24 h at 40° C., then cooled to room temperature. Water (50 mL) was added dropwise, and the mixture extracted with Et₂O (2×200 mL). The combined organic extracts were washed brine, diluted with 10% solution of HCl in dioxane (50 mL) and evaporated under reduced pressure to give 4-benzyl-6,6-difluoro-4-azaspiro[2.4]heptane (3 g, 13.4 mmol, 27% yield).

Step 5: 4-benzyl-6,6-difluoro-4-azaspiro[2.4]heptane (2.68 g, 12 mmol) and palladium hydroxide (0.5 g) in methanol (500 mL) were stirred at room temperature under an atmosphere of H₂ for 24 h. The mixture was filtered and then filtrate concentrated under reduced pressure to obtain 6,6-difluoro-4-azaspiro[2.4]heptane (0.8 g, 6.01 mmol, 50% yield).

### Preparation of 7,7-difluoro-4-azaspiro[2.4]heptane

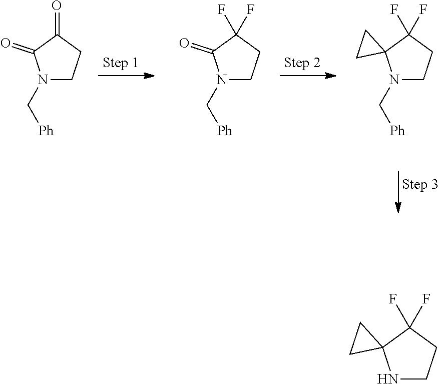 [SMILES: O=C1CCN(Cc2ccccc2)C1=O ; O=C1N(Cc2ccccc2)CCC1(F)F ; FC1(F)CCN(Cc2ccccc2)C12CC2 ; FC1(F)CCNC12CC2]

Step 1: To a cooled (0° C.) solution of 1-benzylpyrrolidine-2,3-dione (8 g, 42.3 mmol) in DCM (100 mL) was added dropwise over 30 minutes DAST (20.4 g, 127 mmol). The mixture was stirred at room temperature overnight, then quenched by dropwise addition of saturated NaHCO₃. The organic layer was separated, and the aqueous fraction extracted twice with DCM (2×50 mL). The combined organic layers were dried over Na₂SO₄ and concentrated under reduced pressure to afford 1-benzyl-3,3-difluoropyrrolidin-2-one (26.0 mmol, 61% yield), which used in the next step without further purification.

Step 2: To a solution of crude 1-benzyl-3,3-difluoropyrrolidin-2-one (5.5 g, 26 mmol) and Ti(Oi-Pr)₄ (23.4 mL, 78 mmol) in THF (300 mL) was added dropwise under argon atmosphere 3.4 M solution of EtMgBr in 2-MeTHF (45.8 mL, 156 mmol). After stirring for 12 h, water (10 mL) was added to obtain a white precipitate. The precipitate was washed with MTBE (3×50 mL). The combined organic fractions were dried over Na₂SO₄, concentrated and purified by flash chromatography (hexanes-EtOAc 9:1) to obtain 4-benzyl-7,7-difluoro-4-azaspiro[2.4]heptane (1.3 g, 5.82 mmol, 22% yield) as a pale yellow oil.

Step 3: 4-benzyl-7,7-difluoro-4-azaspiro[2.4]heptane (0.55 g, 2.46 mmol) was dissolved in solution of CHCl₃ (1 mL) and MeOH (20 mL) and Pd/C (0.2 g, 10%) was added. This mixture was stirred under and an H₂ atmosphere for 5 h, then filtered. The filtrate was concentrated to give 7,7-difluoro-4-azaspiro[2.4]heptane (0.164 g, 1.23 mmol, 50% yield)

### Synthesis of 1-[(difluoromethoxy)methyl]-N-methylcyclopropan-1-amine

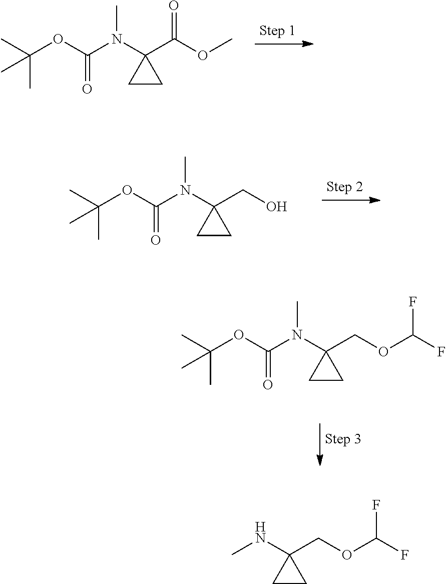

Step 1: To a solution of methyl 1-((tertbutoxycarbonyl)(methyl)amino)cyclopropane-1-carboxylate (1.05 g, 4.58 mmol) in dry THF(5 ml) under N₂ was added lithium borohydride (1.259 ml, 4 M in THF, 5.04 mmol) . The mixture was stirred at rt for 4 days. Sodium sulfate and water were added, the mixture was filtered over a pad of sodium sulfate which was rinsed with dichloromethane. The filtrate was concentrated, to give tert-butyl (1-(hydroxymethyl)cyclopropyl)(methyl)carbamate as a white solid (0.904 g, 95% yield).

Step 2: To a solution of tert-butyl (1-(hydroxymethyl)cyclopropyl)(methyl)carbamate (0.100 g, 0.497 mmol) and (bromodifluoromethyl)trimethylsilane (0.155 ml, 0.994 mmol) in dichloromethane (0.5 ml) was added one drop of a solution of potassium acetate (0.195 g, 1.987 mmol) in water (0.5 ml). The mixture was stirred for 40 h. The mixture was diluted with dichloromethane and water, the organic layer was separated and concentrated. Purifcation by flash chromatography (20% ethyl acetate in heptane) gave a tert-butyl N-{1[(difluoromethoxy)methyl]cyclopropyl}-N-methylcarbamate as colorless oil (0.058 g, 46% yield)

Step 3: To tert-butyl (1-((difluoromethoxy)methyl)cyclopropyl)(methyl)carbamate (0.058 g, 0.231 mmol) was added HCl in dioxane (4M solution, 2 ml, 8.00 mmol). The mixture was stirred for 30 min at rt, then concentrated to yield the desired product which was used without further purification

LC-MS: m/z 152.2 (M+H)+

### Synthesis of tert-butyl 3-{methyl[1-(pyridin-3-yl)cyclopropyl]carbamoyl}-4H,5H,6H,7H-pyrazolo[1,5-a]pyrazine-5-carboxylate

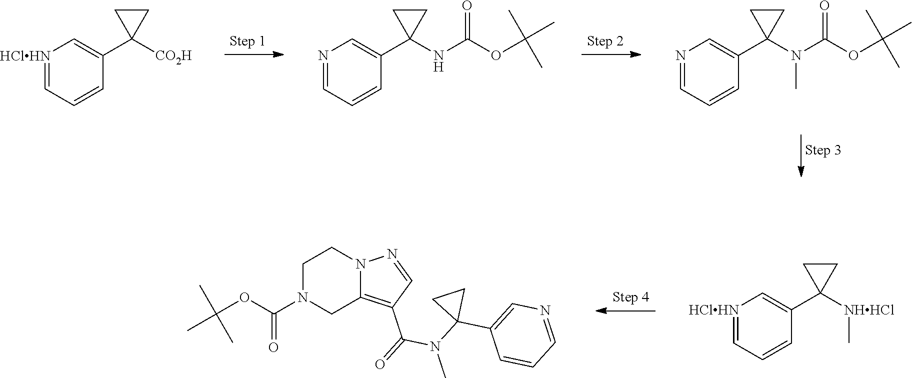

Step 1: To a solution of 1-(pyridin-3-yl)cyclopropane-1-carboxylic acid hydrochloride (498.46 mg, 2.5 mmol) in a mixture of toluene (30 mL) and t-BuOH (10 mL) were added diphenylphosphoryl azide (687.14 mg, 2.5 mmol) and triethylamine (631.62 mg, 6.24 mmol, 870.0 μL). The reaction mixture was heated at reflux overnight. The reaction mixture was cooled and filtered. The filtrate was washed with water (3×10 mL), dried over Na₂SO₄ and concentrated in vacuo to give tert-butyl N-[1-(pyridin-3-yl)cyclopropyl]carbamate (250.0 mg, 95.0% purity, 1.01 mmol, 40.6% yield) as light brown oil.

Step 2: Sodium hydride (154.24 mg, 6.43 mmol) was suspended in dry DMF (5 mL) and then cooled to 0° C. A solution of tert-butyl N-[1-(pyridin-3-yl)cyclopropyl]carbamate (1.51 g, 6.43 mmol) in dry DMF (5 mL) was added dropwise. The resulting mixture was stirred until gas evolution ceased. Iodomethane (1.0 g, 7.07 mmol, 440.0 μl) was added dropwise at that same temperature; the resulting mixture was warmed to r.t. and then stirred overnight. After consumption of the starting material CH NMR control) the reaction mixture was poured into water. The resulting mixture was extracted twice with MTBE (2×50 mL). The organic phases were combined, washed with water, dried over sodium sulfate and concentrated to give tert-butyl N-methyl-N-[1-(pyridin-3-yl)cyclopropyl]carbamate (1.1 g, 4.43 mmol, 68.9% yield). The product was used in the next step without further purification.

Step 3: To a solution of tert-butyl N-methyl-N-[1-(pyridin-3-yl)cyclopropyl]carbamate (1.1 g, 4.43 mmol) in methanol (10 mL) was added 4M HCl solution in dioxane (2 mL). The resulting solution was stirred for 12 h at 25° C. Upon completion of the reaction (monitored by ¹H NMR or LCMS), the reaction mixture was concentrated under reduced pressure. The product was triturated with MTBE and collected by filtration, then dried in vacuo at 40° C., to give N-methyl-1-(pyridin-3-yl)cyclopropan-1-amine dihydrochloride (900.0 mg, 95.0% purity, 3.87 mmol, 87.2% yield).

Step 4: To a stirred solution of N-methyl-1-(pyridin-3-yl)cyclopropan-l-amine dihydrochloride (398.89 mg, 1.8 mmol) and 5-[(tert-butoxy)carbonyl]-4H,5H,6H,7H-pyrazolo[1,5-a]pyrazine-3-carboxylic acid (482.15 mg, 1.8 mmol) in DMF (2 mL) were added HATU (891.67 mg, 2.35 mmol) and triethylamine (638.88 mg, 6.31 mmol, 880.0 μl). The mixture was stirred overnight at r.t. and then poured onto water and extracted with MTBE (2×15 mL). The combined organic fractions were washed three times with water, dried over anhydrous sodium sulfate, and the solvent was removed in vacuum. The crude product was purified by HPLC to give tert-butyl 3-methyl[1-(pyridin-3-yl)cyclopropyl]carbamoyl-4H,5H, 6H,7H-pyrazolo[1,5-a]pyrazine-5-carboxylate (230.0 mg, 82.0% purity, 474.5 μmol, 26.3% yield).

¹H NMR (400 MHz, d6-DMSO) δ 1.41; (m, 2H), 1.43; (s, 9H), 1.56; (m, 2H), 3.07; (m, 3H), 3.82; (m, 2H), 4.07; (m, 2H), 4.75; (m, 2H), 6.99; (m, 1H), 7.37; (m, 1H), 7.48; (d, 1H), 8.31; (s, 1H), 8.44; (s, 1H).

LCMS: m/z 398.2

### Synthesis of tert-butyl 3-{methyl[1-(pyridin-4-yl)cyclopropyl]carbamoyl}-4H,5H,6H,7H-pyrazolo[1,5-a]pyrazine-5-carboxylate

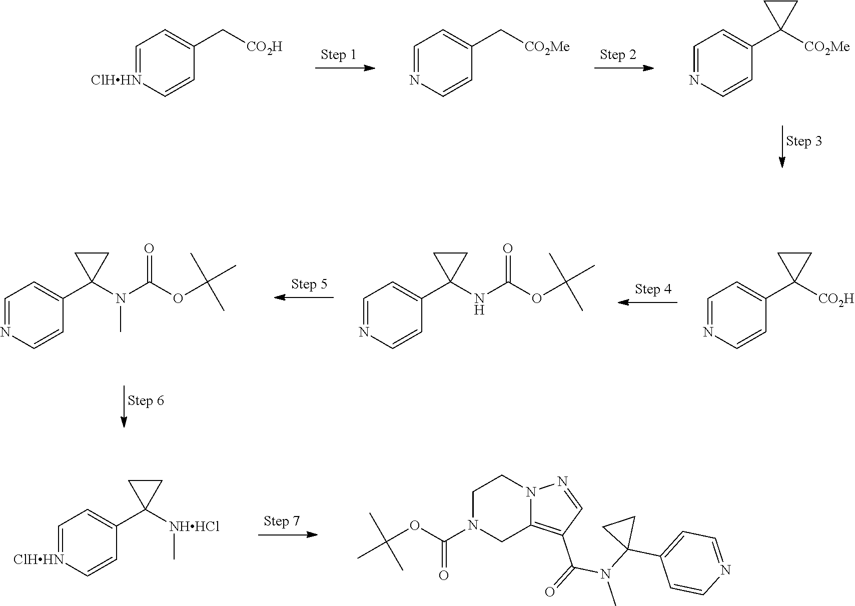

Step 1: 2-(Pyridin-4-yl)acetic acid hydrochloride (5.0 g, 28.8 mmol) was dissolved in Me0H (20 mL), then H₂SO₄ (0.5 mL) was added. The reaction mixture was heated at 85° C. overnight. The MeOH was removed to give a residue which was carefully neutralized with saturated aqueous NaHCO₃ solution and then extracted with EtOAc (3×100 mL). The organic extracts were combined, dried and concentrated to give methyl 2-(pyridin-4-yl)acetate (4.0 g, 95.0% purity, 25.14 mmol, 87.3% yield) as a yellow oil, which was used in the next step without further purification.

Step 2: Methyl 2-(pyridin-4-yl)acetate (4.0 g, 26.46 mmol) was dissolved in DMF (5 mL) and added dropwise to a cooled (0° C.) suspension of sodium hydride (825.52 mg, 34.4 mmol) in DMF (5 mL). The resulting mixture was stirred at 0° C. for 30 min and then treated with 1,2-dibromoethane (6.46 g, 34.4 mmol) at the same temperature. The reaction mixture was stirred at r.t. for 12 h. The reaction mixture was then diluted with ethyl acetate and washed with water and brine. The organic phase was separated, dried over Na₂SO₄ and filtered; the filtrate was concentrated. The resulting oil was triturated with hexane to give methyl 1-(pyridin-4-yl)cyclopropane-1-carboxylate (2.3 g, 12.98 mmol, 49.1% yield) as a solid.

Step 3: Methyl 1-(pyridin-4-yl)cyclopropane-1-carboxylate (2.3 g, 12.98 mmol) was dissolved in MeOH (20 mL), to which was added a solution of sodium hydroxide (778.67 mg, 19.47 mmol) in water (20 mL). The mixture was stirred at 20° C. for 20 h. MeOH was removed by evaporation and the aqueous residue was neutralized under ice cooling with hydrochloric acid (to pH 7). The mixture was concentrated to dryness, the residue was triturated three times with CHCl₃, and the combined filtrates concentrated to dryness to give 1-(pyridin-4-yl)cyclopropane-1-carboxylic acid hydrochloride (2.0 g, 10.02 mmol, 77.2% yield).

Step 4: To solution of 1-(pyridin-4-yl)cyclopropane-1-carboxylic acid (599.43 mg, 3.67 mmol) in mixture of toluene (30 mL) and t-BuOH (10 mL) were added diphenylphosphoryl azide (1.01 g, 3.67 mmol) and triethylamine (929.28 mg, 9.18 mmol, 1.28 mL). The reaction mixture was refluxed overnight, then cooled and filtered. The filtrate was washed with water (3×10 mL), dried over Na₂SO₄ and concentrated to give tert-butyl N[1-(pyridin-4-yl)cyclopropyl]carbamate (300.0 mg, 1.28 mmol, 34.9% yield) as light brown oil. The product was used in the next step without further purification.

Step 5: Sodium hydride (94.22 mg, 3.93 mmol) was suspended in DMF (5 mL) and then cooled to 0° C. A solution of tert-butyl N[1-(pyridin-4-yl)cyclopropyl]carbamate (919.93 mg, 3.93 mmol) in DMF (5 mL) was then added dropwise. The resulting mixture was stirred until gas evolution ceased. Iodomethane (613.04 mg, 4.32 mmol) was added dropwise at that same temperature; the resulting mixture was warmed to r.t. and then stirred overnight. After consumption of the starting material (¹H NMR control) the reaction mixture was poured into water. The mixture was extracted twice with MTBE (50 mL). The organic phases were combined, washed with water, dried over sodium sulfate and concentrated to give tert-butyl N-methyl-N-[1-(pyridin-4-yl)cyclopropyl]carbamate (900.0 mg, 98.0% purity, 3.55 mmol, 90.5% yield). The product was used in the next step without further purification.

Step 6: To a solution of tert-butyl N-methyl-N-[1-(pyridin-4-yl)cyclopropyl]carbamate (900.0 mg, 3.62 mmol) in methanol (10 mL) was added 4M HCl in dioxane (2 mL) and the resulting solution was stirred for 12 h at 25° C. Upon completion of the reaction (monitored by ¹H NMR), the reaction mixture was concentrated under reduced pressure. The product was treated with MTBE and collected by filtration, then dried in vacuo at 40° C., to give N-methyl-1-(pyridin-4-yl)cyclopropan-1-amine dihydrochloride (600.0 mg, 2.71 mmol, 74.9% yield).

Step 7: To a stirred solution of N-methyl-1-(pyridin-4-yl)cyclopropan-1-amine dihydrochloride (600.0 mg, 2.71 mmol) and 5-[(tert-butoxy)carbonyl]-4H,5H,6H,7H-pyrazolo[1,5-a]pyrazine-3-carboxylic acid (724.91 mg, 2.71 mmol) in DMF (5 mL) were added HATU (1.34 g, 3.53 mmol) and triethylamine (960.55 mg, 9.49 mmol, 1.32 ml) . The mixture was stirred overnight at r.t. and then poured into water and extracted with MTBE (3×15 mL). The combined organic fractions were washed three times with water, dried over anhydrous sodium sulfate, and concentrated. The crude product was purified by HPLC to give tert-butyl 3-methyl[1-(pyridin-4-yl)cyclopropyl]carbamoyl-4H,5H,6H,7H-pyrazolo[1,5-a]pyrazine-5-carboxylate (169.0 mg, 425.19 μmol, 15.7% yield).

¹H NMR (400 MHz, d6-DMSO) δ 1.38; (m, 1H), 1.44; (s, 9H), 1.60; (m, 3H), 3.03; (m, 3H), 3.71; (m, 1H), 3.84; (m, 1H), 4.06; (m, 2H), 4.75; (m, 2H), 6.92; (m, 1H), 7.07; (m, 2H), 8.52; (m, 2H).

LCMS: m/z 398.4

### Synthesis of tert-butyl 3-{methyl[1-(pyrimidin-2-yl)cyclopropyl]carbamoyl}-4H,5H,6H,7H-pyrazolo[1,5-a]pyrazine-5-carboxylate

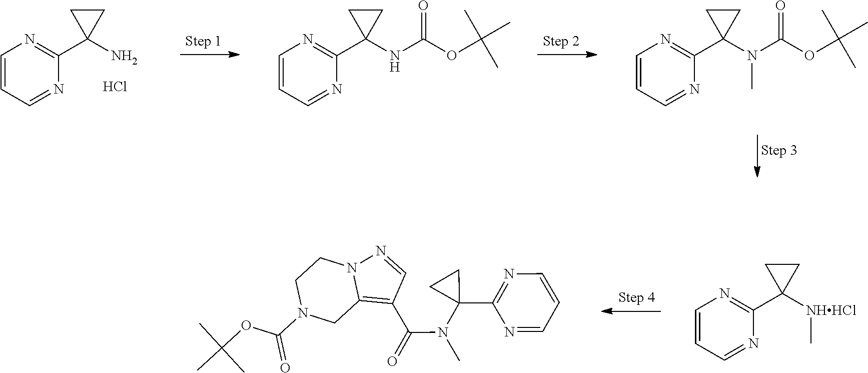

Step 1: To a cooled (0° C.) suspension of 1-(pyrimidin-2-yl)cyclopropan-1-amine hydrochloride (996.43 mg, 5.81 mmol) in dry DCM (30 mL) was added di-tert-butyl dicarbonate (1.27 g, 5.81 mmol). Triethylamine (646.14 mg, 6.39 mmol, 890.0 μL) was then added dropwise. The reaction mixture was stirred overnight at r.t and diluted with water (5 mL). The organic phase was separated, washed with water, dried over sodium sulfate, filtered and concentrated to afford tert-butyl N[1-(pyrimidin-2-yl)cyclopropyl]carbamate (1.17 g, 4.97 mmol, 85.7% yield) as a light yellow solid.

Step 2: To a stirred solution of tert-butyl n-[1-(pyrimidin-2-yl)cyclopropyl]carbamate (499.99 mg, 2.13 mmol) in dry DMF (4 mL) was added sodium hydride (127.49 mg, 5.31 mmol). The reaction mixture was stirred at r.t. for 1 h, then cooled to 0° C. Iodomethane (603.26 mg, 4.25 mmol) was added. The mixture was stirred at r.t. overnight. The mixture was poured into brine; then iextracted with EtOAc (2×10 mL). The combined organic phases were washed with brine, dried over Na₂SO₄, filtered and concentrated to afford tert-butyl N-methyl-N-[1-(pyrimidin-2-yl)cyclopropyl]carbamate (400.0 mg, 1.6 mmol, 75.5% yield) as yellow solid.

Step 3: To a stirred solution of tert-butyl N-methyl-N-[1-(pyrimidin-2-yl)cyclopropyl]carbamate (400.0 mg, 1.6 mmol) in dry DCM (5 mL) was added 4M HCl in dioxane (2 mL, 8 mmol). The reaction mixture was stirred at r.t. for 5 h. The mixture was concentrated, the residue was triturated with hexane and filtered off to afford N-methyl-1-(pyrimidin-2-yl)cyclopropan-1-amine hydrochloride (280.0 mg, 1.51 mmol, 94% yield) as grey solid.

Step 4: To a cooled (0° C.) solution of HATU (573.46 mg, 1.51 mmol) and 5-[(tert-butoxy)carbonyl]-4H,5H,6H,7H-pyrazolo[1,5-a]pyrazine-3-carboxylic acid (403.11 mg, 1.51 mmol) in DMF (3 mL) were added successively N-methyl-1-(pyrimidin-2-yl)cyclopropan-1-amine hydrochloride (280.0 mg, 1.51 mmol) and N,N-diisopropylethylamine (779.69 mg, 6.03 mmol) dropwise. The reaction mixture was stirred at r.t. overnight and diluted with brine. The mixture was extracted with EtOAc (2×10 mL), the combined organic phases were washed with brine, dried over Na₂SO₄ and concentrated. The residue was purified by HPLC to give tert-butyl 3-methyl[1-(pyrimidin-2-yl)cyclopropyl]carbamoyl-4H,5H,6H,7H-pyrazolo[1,5-a]pyrazine-5-carboxylate (332.9 mg, 835.47 μmol, 55.4% yield) as yellow solid.

¹H NMR (400 MHz, d6-DMSO) δ 1.43; (s, 9H), 1.57; (m, 2H), 1.89; (m, 1H), 3.31; (m, 2H), 3.71; (m, 1H), 3.83; (m, 2H), 4.03; (m, 2H), 4.12; (m, 1H), 4.69; (m, 1H), 4.78; (m, 1H), 6.78; (s, 1H), 7.36; (t, 1H), 8.78; (d, 2H).

LCMS: m/z 399.2

### Synthesis of tert-butyl 3-[(1-{[(2,2-difluoroethyl)amino]methyl}cyclopropyl)(methyl)carbamoyl]-4H,5H,6H,7H-pyrazolo[1,5-a]pyrazine-5-carboxylate

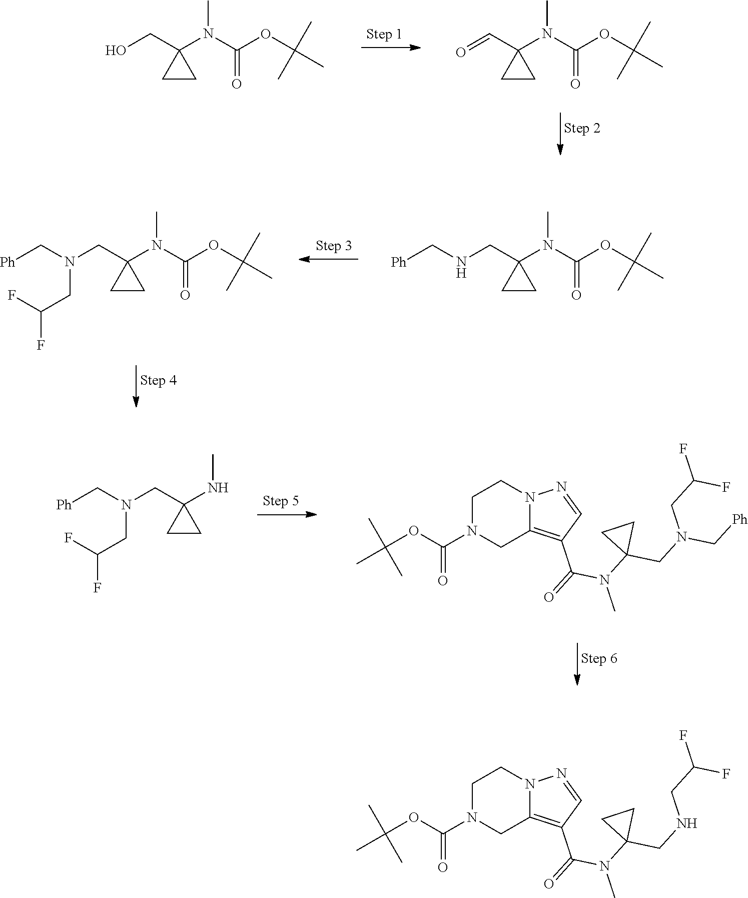

Step 1: To a stirred solution of tert-butyl N-[1-(hydroxymethyl)cyclopropyl]-N-methylcarbamate (2.25 g, 11.18 mmol) in dry DCM (30 mL) at r.t. was added 1,1,1-tris(acetoxy)-1,1-dihydro-1,2-benziodoxol-3(1H)-one (4.74 g, 11.18 mmol) portionwise. The reaction mixture was stirred at r.t. for 1 h and then cooled to 0° C. A solution of sodium hydroxide (2.01 g, 50.3 mmol) in water (5 mL) was then added dropwise and the mixture was stirred at r.t. for 15 min. The organic phase was separated, dried over Na₂SO₄, filtered and concentrated to afford tert-butyl N-(1-formylcyclopropyl)-N-methylcarbamate (2.2 g, 11.04 mmol, 98.8% yield) as yellow oil.

Step 2: To a stirred solution of tert-butyl N-(1-formylcyclopropyl)-N-methylcarbamate (2.2 g, 11.04 mmol) in dry DCM (50 mL) was added phenylmethanamine (1.18 g, 11.04 mmol). The mixture was stirred at r.t. for 5 h. To the cooled reaction mixture was added sodium bis(acetyloxy)boranuidyl acetate (7.02 g, 33.12 mmol) in one portion and stirring was continued for 5 h. The mixture was cooled to 0° C. and 15% aq. solution of NaOH (20 mL) was added. The mixture was stirred for 30 min and organic phase was separated, dried over Na₂SO₄, filtered and concentrated to afford tert-butyl N-1-[(benzylamino)methyl]cyclopropyl-N-methylcarbamate (2.75 g, 85% yield) as yellow oil.

Step 3: To a stirred, cooled (0° C.) solution of tert-butyl N-1-[(benzylamino)methyl]cyclopropyl-N-methylcarbamate (1.75 g, 6.02 mmol) in dry acetonitrile (10 mL) was added potassium carbonate (1.67 g, 12.05 mmol) followed by dropwise addition of 2,2-difluoroethyl trifluoromethanesulfonate (1.68 g, 7.83 mmol). The reaction mixture was warmed to r.t. and stirred overnight. The mixture was poured into water (30 mL) and extracted with DCM (3×10 mL). The combined organic phases was dried over Na₂SO₄, filtered and concentrated. The residue was purified by flash column chromatography on silica with hexane-MTBE (4:1) as eluent to afford tert-butyl N-(1-[benzyl(2,2-difluoroethyl)amino]methylcyclopropyl)-N-methylcarbamate (900.0 mg, 2.54 mmol, 42.2% yield) as colorless oil.

Step 4: To a solution of tert-butyl N-(1-[benzyl(2,2-difluoroethyl)amino]methylcyclopropyl)-N-methylcarbamate (199.9 mg, 564.0 μmol) in CH₂Cl₂ (3 mL) was added 4M HCl in dioxane (1 mL). The resulting solution was stirred for 12 h at r.t., then concentrated. The residue was triturated with hexane and collected by filtration, to give 1-[benzyl(2,2-difluoroethyl)amino]methyl-N-methylcyclopropan-1-amine dihydrochloride (156.0 mg, 95.1% yield) as white solid.

Step 5: To a solution of 1-[benzyl(2,2-difluoroethyl)amino]methyl-N-methylcyclopropan-1-amine dihydrochloride (155.96 mg, 476.58 μmol) and [(dimethylamino)(3H-[1,2,3]triazolo[4,5-b]pyridin-3-yloxy)methylidene]dimethylazanium; hexafluoro-lambda5-phosphanuide (181.21 mg, 476.58 μmol) in DMF (2 mL) was added triethylamine (241.13 mg, 2.38 mmol). The mixture was stirred at r.t. for 15 mins. 5-[(Tert-butoxylcarbonyl]-4H,5H,6H,7H-pyrazolo[1,5-a]pyrazine-3-carboxylic acid (127.38 mg, 476.58 μmol) was added, and the reaction stirred at r.t. for 24 h, then diluted with brine. The mixture was extracted with EtOAc (2×20 mL). The combined organic phases was washed with brine, dried over Na₂SO₄, filtered and concentrated to afford crude tert-butyl 3-[(1-[benzyl(2,2-difluoroethyl)amino]methylcyclopropyl)(methyl)carbamoyl]-4H,5H,6H,7H-pyrazolo[1,5-a]pyrazine-5-carboxylate (200.0 mg, 397.15 μmol, 83.3% yield) as brown oil that was used in the next step without further purification. Step 6: To a stirred solution of tert-butyl 3-[(1-[benzyl(2,2-difluoroethyl)amino]methylcyclopropyl)(methyl)carbamoyl]-4H,5H,6H,7H-pyrazolo[1,5-a]pyrazine-5-carboxylate (200.0 mg, 397.15 μmol) in MeOH (5 mL) was added palladium on carbon (10%, 0.05 g). The mixture was stirred at r.t. under hydrogen (balloon) for 48 h. The mixture was purged with nitrogen, then filtered, and the filtrate concentrated. The residue was purified by HPLC to give tert-butyl 3-[(1-[(2,2 difluoroethyl)amino]methylcyclopropyl)(methyl)carbamoyl]-4H,5H,6H,7H-pyrazolo[1,5-a]pyrazine-5-carboxylate (70.0 mg, 42.7% yield) as colorless oil.

¹H NMR (400 MHz, d6-DMSO) δ 0.76; (m, 3H), 1.43; (s, 9H), 2.26; (m, 1H), 2.90; (m, 4H), 3.05; (s, 3H), 3.80; (s, 2H), 4.10; (d, 2H), 4.71; (s, 2H), 5.96; (tt, 1H), 7.84; (s, 1H).

LCMS: m/z 414.1

### Synthesis of tert-butyl 3-[methyla-{[(2,2,2-trifluoroethyl)amino]methyl}cyclopropyl)carbamoyl]-4H,5H,6H,7H-pyrazolo[1,5-a]pyrazine-5-carboxylate

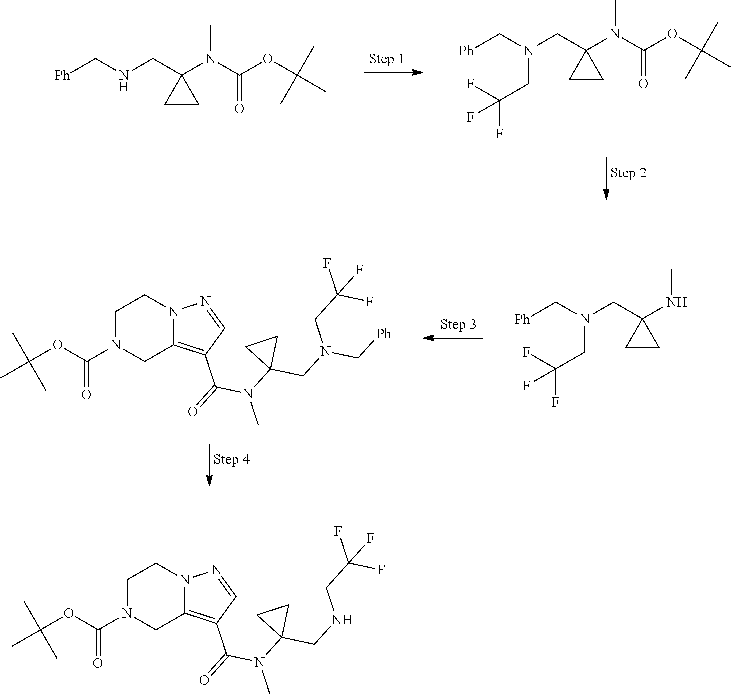 [SMILES: CN(C(=O)OC(C)(C)C)C1(CNCc2ccccc2)CC1 ; CN(C(=O)OC(C)(C)C)C1(CN(Cc2ccccc2)CC(F)(F)F)CC1 ; CN(C(=O)c1cnn2c1CN(C(=O)OC(C)(C)C)CC2)C1(CN(Cc2ccccc2)CC(F)(F)F)CC1 ; CNC1(CN(Cc2ccccc2)CC(F)(F)F)CC1 ; CN(C(=O)c1cnn2c1CN(C(=O)OC(C)(C)C)CC2)C1(CNCC(F)(F)F)CC1]

Step 1: To a stirred solution of tert-butyl N-1-[(benzylamino)methyl]cyclopropyl-N-methylcarbamate (537.25 mg, 1.85 mmol) in dry acetonitrile (10 mL) was added potassium carbonate (767.06 mg, 5.55 mmol) followed by 2,2,2-trifluoroethyl trifluoromethanesulfonate (644.56 mg, 2.78 mmol, 400.0 μL). The reaction mixture was stirred at 80° C. overnight. The mixture was then cooled, concentrated, and the residue obtained was dissolved in DCM (10 mL). The organic phase was washed with water (3 mL), dried over Na₂SO₄ and concentrated. The residue was purified by flash column chromatography on (hexane-MTBE 10:1) to afford tert-butyl N-(1-[benzyl (2,2,2-trifluoroethyl)amino]methylcyclopropyl)-N-methylcarbamate (410.0 mg, 1.1 mmol, 59.5% yield) as colorless oil.

Step 2: To a stirred solution of tert-butyl N-(1-[benzyl(2,2,2-trifluoroethyl)amino]methylcyclopropyl)-N-methylcarbamate (410.0 mg, 1.1 mmol) in DCM (5 mL) was added 4M HCl in dioxane (3 mL, 12 mmol). The resulting mixture was stirred overnight, then evaporated to dryness to give 1-[benzyl(2,2,2-trifluoroethyl)amino]methyl-N-methylcyclopropan-1-amine dihydrochloride (330.0 mg, 955.88 nmol, 86.8% yield) as yellow oil.

Step 3: To a solution of HATU (381.96 mg, 1.0 mmol) in DMF (3 mL) were added triethylamine (484.05 mg, 4.78 mmol) and 5-[(tert-butoxy)carbonyl]-4H,5H,6H,7H-pyrazolo[1,5-a]pyrazine-3-carboxylic acid (255.71 mg, 956.72 μmol). The reaction mixture was stirred at r.t. for 30 mins, then a solution of 1-[benzyl(2,2,2-trifluoroethyl)amino]methyl-N-methylcyclopropan-1-amine dihydrochloride (330.29 mg, 956.72 μmol) in DMF (1 mL) was added. The reaction mixture was stirred at r.t. overnight and poured into water (5 mL). The mixture was extracted with EtOAc (2×5 mL). The combined organic phases was washed with water, aq. NaHCO₃, dried over Na₂SO₄, filtered and concentrated to afford crude tert-butyl 3-[(1-[benzyl(2,2,2-trifluoroethyl)amino]methylcyclopropyl)(methyl)carbamoyl]-4H,5H,6H,7H-pyrazolo[1,5-a]pyrazine-5-carboxylate (600.0 mg, 77.0% purity, 885.78 μmol, 92.6% yield) as brown oil, that was used in the next step without further purification.

Step 4: To a stirred solution of tert-butyl 3-[(1-[benzyl(2,2,2-trifluoroethyl)amino]methylcyclopropyl)(methyl)carbamoyl]-4H,5H,6H,7H-pyrazolo[1,5-a]pyrazine-5-carboxylate (600.0 mg, 1.15 mmol) in MeOH (10 mL) was added palladium on carbon (10%, 70 mg). The mixture was stirred under H₂ (balloon) for 5 days. The mixture was filtered, concentrated, and purified by HPLC to give tert-butyl 3-[methyl(1-[(2,2,2-trifluoroethyl)amino]methylcyclopropyl)carbamoyl]-4H,5H,6H,7H-pyrazolo[1,5-a]pyrazine-5-carboxylate (218.5 mg, 506.43 nmol, 44.1% yield) as brown oil.

¹H NMR (400 MHz, d6-DMSO) δ 0.76; (s, 3H), 1.43; (s, 9H), 2.65; (m, 1H), 2.90; (m, 1H), 3.11; (m, 3H), 3.27; (m, 3H), 3.80; (m, 2H), 4.10; (m, 2H), 4.71; (m, 2H), 7.83; (m, 1H).

LCMS: m/z 432.2

### Synthesis of 4-{4H,5H,6H,7H-pyrazolo[1,5-a]pyrazine-3-carbonyl}-8-oxa-4-azaspiro[2.6]nonane

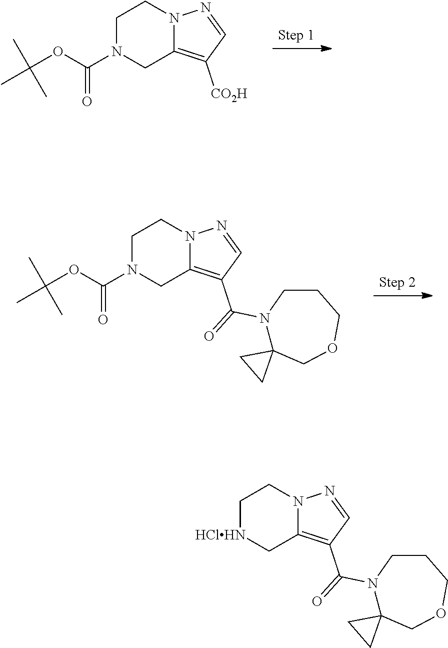

Step 1: To a stirred solution of 5-[(tert-butoxy)carbonyl]-4H,5H,6H,7H-pyrazolo [1,5-a]pyrazine-3-carboxylic acid (489.9 mg, 1.83 mmol) and 8-oxa-4-azaspiro[2.6]nonane hydrochloride (300.0 mg, 1.83 mmol) in DMF (5 mL) were added HATU (906.01 mg, 2.38 mmol) and triethylamine (649.15 mg, 6.42 mmol, 890.0 μL). Schem The mixture was stirred overnight at r.t. and then poured into water and extracted with MTBE (2×15 mL). The combined organic fractions were washed three times with water (20 mL), dried over Na₂SO₄, and concentrated to give tert-butyl 3-8-oxa-4-azaspiro[2.6]nonane-4-carbonyl-4H,5H,6H,7H-pyrazolo[1,5-a]pyrazine-5-carboxylate (500.0 mg, 91.0% purity, 1.21 mmol, 65.9% yield).

Step 2: To a solution of tert-butyl 3-8-oxa-4-azaspiro[2.6]nonane-4-carbonyl-4H,5H,6H,7H-pyrazolo[1,5-a]pyrazine-5-carboxylate (500.0 mg, 1.33 mmol) in MeOH (10 mL) was added 4M HCl in dioxane (2 mL, 8 mmol). The resulting solution was stirred for 12 h, and then concentrated under reduced pressure. The product was treated with MTBE (50 mL) and collected by filtration, then dried in vacuo at 40° C., to give 4-4H,5H,6H,7H-pyrazolo[1,5-a]pyrazine-3-carbonyl-8-oxa-4-azaspiro[2.6]nonane hydrochloride (220.0 mg, 90.0% purity, 633.0 μmol, 54% yield).

¹H NMR (500 MHz, d6-DMSO) δ 0.90; (m, 4H), 1.95; (m, 2H), 3.50; (m, 3H), 3.64; (m, 5H), 4.37; (m, 2H), 4.47; (m, 2H), 7.77; (s, 1H), 10.09; (m, 2H).

LCMS: m/z 277.2

### Synthesis of 4-{4H,5H,6H,7H-pyrazolo[1,5-a]pyrazine-3-carbonyl}-7-oxa-4-azaspiro[2.6]nonane

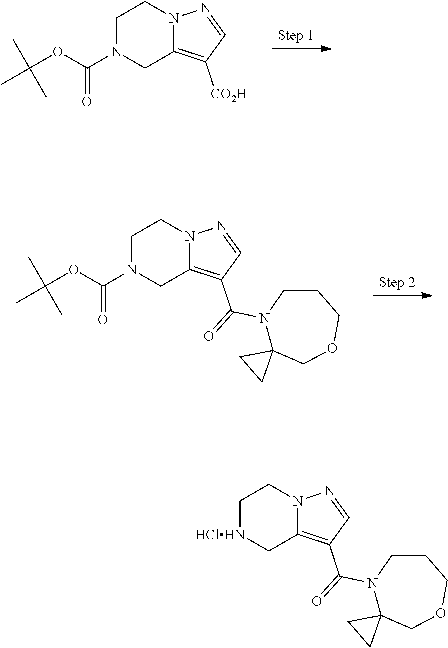

Step 1: To a stirred solution of 5-[(tert-butoxy)carbonyl]-4H,5H,6H,7H-pyrazolo[1,5-a]pyrazine-3-carboxylic acid (489.9 mg, 1.83 mmol) and 7-oxa-4-azaspiro[2.6]nonane hydrochloride (300.0 mg, 1.83 mmol) in DMF (5 mL) were added HATU (906.01 mg, 2.38 mmol) and triethylamine (649.15 mg, 6.42 mmol, 890.0 μL). The mixture was stirred overnight at r.t. and then poured into water and extracted with MTBE (2×15 mL). The combined organic fractions were washed three times with water, dried over anhydrous sodium sulfate, and concentrated to give tert-butyl 3-7-oxa-4-azaspiro[2.6]nonane-4-carbonyl-4H,5H,6H,7H-pyrazolo[1,5-a]pyrazine-5-carboxylate (350.0 mg, 95.0% purity, 883.25 μmol, 48.2% yield).

Step 2: To a solution of tert-butyl 3-7-oxa-4-azaspiro[2.6]nonane-4-carbonyl-4H,5H,6H,7H-pyrazolo[1,5-a]pyrazine-5-carboxylate (350.0 mg, 929.74 μmol) in methanol (10 ml) was added 4N HCl solution in dioxane (2 mL) and the resulting solution was stirred for 12 h at 25° C. Upon completion of the reaction (monitored by HNMR), the reaction mixture was concentrated under reduced pressure. The product was treated with MTBE and collected by filtration, then dried in vacuo at 40° C., to give 4-4H,5H,6H,7H-pyrazolo[1,5-a]pyrazine-3-carbonyl-7-oxa-4-azaspiro[2.6]nonane hydrochloride (110.0 mg, 91.0% purity, 320.02 μmol, 34.4% yield).

¹H NMR (400 MHz, D₂O) δ 0.87; (m, 4H), 1.73; (m, 1H), 3.71; (m, 5H), 3.93; (m, 2H), 4.39; (m, 2H), 4.55; (m, 3H), 7.82; (m, 1H).

LCMS: m/z 277.2

### Synthesis of tert-butyl 3-{7-hydroxy-4-azaspiro[2.5]octane-4-carbonyl}-4H,5H,6H,7H-pyrazolo[1,5-a]pyrazine-5-carboxylate

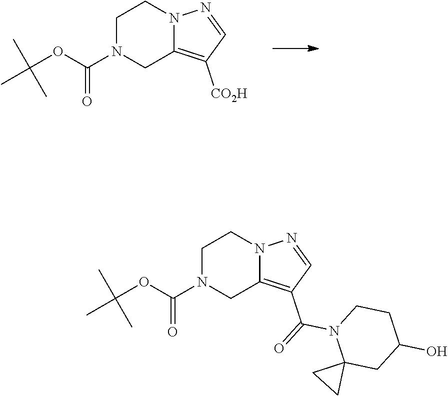 [SMILES: CC(C)(C)OC(=O)N1CCn2ncc(C(=O)O)c2C1 ; CC(C)(C)OC(=O)N1CCn2ncc(C(=O)N3CCC(O)CC34CC4)c2C1]

To a solution of 5-[(tert-butoxy)carbonyl]-4H,5H,6H,7H-pyrazolo[1,5-a]pyrazine-3-carboxylic acid (1.13 g, 4.22 mmol) and triethylamine (1.07 g, 10.55 mmol, 1.47 ml) in MeCN (20 mL) was added HATU (1.77 g, 4.64 mmol). The resulting mixture was stirred for 10 min then 4-azaspiro[2.5]octan-7-ol hydrochloride (760.0 mg, 4.64 mmol) was added and the stirring was continued overnight. The reaction mixture was partitioned between EtOAc (50 mL) and water (100 mL). The organic phase was washed with water (2×20 mL), brine, dried over sodium sulfate and concentrated under reduced pressure. The product was purified by HPLC to give tert-butyl 3-7-hydroxy-4-azaspiro[2.5]octane-4-carbonyl-4H, 5H,6H,7H-pyrazolo[1,5-a]pyrazine-5-carboxylate (275.0 mg, 730.51 μmol, 17.3% yield).

¹NMR (400 MHz, d6-DMSO) δ 0.56; (m, 2H), 0.82; (m, 1H), 0.92; (m, 1H), 1.20; (m, 1H), 1.43; (s, 9H), 1.81; (m, 2H), 3.75; (m, 1H), 3.83; (m, 3H), 4.11; (m, 4H), 4.62; (m, 1H), 4.71; (m, 1H), 4.76; (m, 1H), 7.70; (s, 1H).

LCMS: m/z 377.2

### Example 1

N5-(3-chloro-4-fluorophenyl)-N3-[1-(methoxymethyl)cyclopropyl]-N3,6-dimethyl-4H,5H,6H,7H-pyrazolo[1,5-a]pyrazine-3,5-dicarboxamide

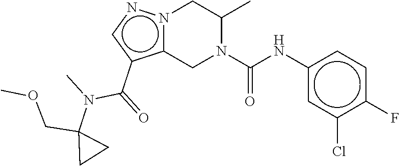

Rt (Method A) 3.16 mins, m/z 450/452 [M+H]+

¹H NMR (400 MHz, DMSO-d6) δ 9.01; (s, 1H), 8.05-7.80; (m, 1H), 7.74; (dd, J=6.9, 2.6 Hz, 1H), 7.45-7.39; (m, 1H), 7.31; (t, J=9.1 Hz, 1H), 5.39-5.10; (m, 1H), 4.98-4.78; (m, 1H), 4.55-4.35; (m, 1H), 4.27-4.19; (m, 1H), 4.13; (d, J=12.9 Hz, 1H), 3.65-3.45; (m, 2H), 3.29; (s, 3H), 3.23-2.87; (m, 3H), 1.25-0.67; (m, 7H).

### Example 2

N5-(3-chloro-4-fluorophenyl)-N3-methyl-N3-{1-[(propan-2-yloxy)methyl]cyclopropyl}-4H,5H,6H,7H-pyrazolo[1,5-a]pyrazine-3,5- dicarboxamide

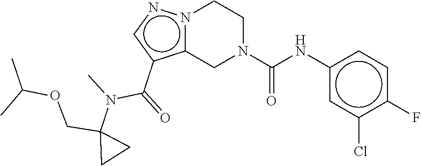

Rt (Method J) 1.51 mins, m/z 464/466 [M+H]+

¹H NMR (400 MHz, DMSO-d6) δ 9.07; (s, 1H), 7.99; (s, 1H), 7.72; (dd, J=6.8, 2.6 Hz, 1H), 7.41; (ddd, J=9.0, 4.3, 2.6 Hz, 1H), 7.31; (t, J=9.1 Hz, 1H), 4.85; (m, 2H), 4.17; (m, 2H), 4.13-3.68; (m, 2H), 3.56; (m, 3H), 3.04; (m, 3H), 1.07; (m, 7H), 0.81; (m, 3H).

### Example 3

N5-(3-chloro-4-fluorophenyl)-N3-[1-(ethoxymethyl)cyclopropyl]-N3-methyl-4H,5H,6H,7H-pyrazolo[1,5-a]pyrazine-3,5-dicarboxamide

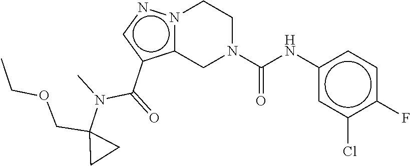

Rt (Method J) 1.42 mins, m/z 450/452 [M+H]+

¹H NMR (400 MHz, DMSO-d6) δ 9.07; (s, 1H), 7.95; (s, 1H), 7.73 (dd, J=6.8, 2.6 Hz, 1H), 7.41; (ddd, J=9.1, 4.3, 2.6 Hz, 1H), 7.31; (t, J=9.1 Hz, 1H), 4.85; (m, 2H), 4.18; (t, J=5.5 Hz, 2H), 3.98; (m, 2H), 3.58; (m, 2H), 3.46; (q, J=7.0 Hz, 2H), 3.05; (m, 3H), 1.10; (m, 4H), 0.83; (s, 3H).

### Example 4

N5-(3-chloro-4-fluorophenyl)-N3-{1-[(difluoromethoxy)methyl]cyclopropyl}-N3-methyl-4H,5H,6H,7H-pyrazolo[1,5-a]pyrazine-3,5-dicarboxamide

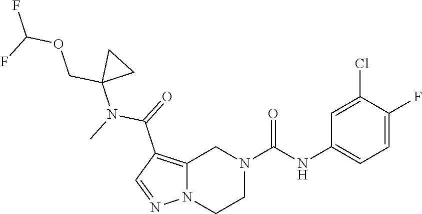 [SMILES: CN(C(=O)c1cnn2c1CN(C(=O)Nc1ccc(F)c(Cl)c1)CC2)C1(COC(F)F)CC1]

Rt (Method B) 3.24 mins, m/z 472/474 [M+H]+

¹H NMR (400 MHz, DMSO-d6) δ 9.08; (s, 1H), 7.82; (s, 1H), 7.73; (dd, J=6.9, 2.6 Hz, 1H), 7.41; (ddd, J=9.0, 4.4, 2.6 Hz, 1H), 7.31; (t, J=9.1 Hz, 1H), 6.71; (t, J=75.8 Hz, 1H), 4.90-4.81; (m, 2H), 4.21-4.14; (m, 2H), 4.11-3.82; (m, 4H), 3.20-2.98; (m, 3H), 1.20-0.79; (m, 4H).

### Example 5

N-(3-chloro-4-fluorophenyl)-3-{6,6-difluoro-4-azaspiro[2.4]heptane-4-carbonyl}-4H,5H,6H,7H-pyrazolo[1,5-a]pyrazine-5-carboxamide

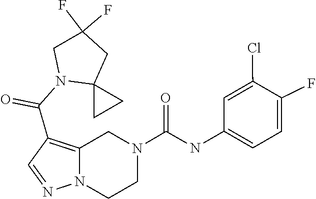

Rt (Method J) 1.47 mins, m/z 454/456 [M+H]+

¹H NMR (400 MHz, DMSO-d6) δ 9.08; (s, 1H), 7.86; (s, 1H), 7.75-7.69; (m, 1H), 7.44-7.38; (m, 1H), 7.31; (t, J=9.1 Hz, 1H), 4.83; (m, 2H), 4.27; (t, J=13.1 Hz, 2H), 4.22-4.12; (m, 2H), 3.98-3.87; (m, 2H), 2.50-2.43; (m, 2H), 1.92-1.84; (m, 2H), 0.69-0.62; (m, 2H).

### Example 6

N5-(3-chloro-4-fluorophenyl)-N3-{1-[(difluoromethoxy)methyl]cyclopropyl}-N3,6-dimethyl-4H,5H,6H,7H-pyrazolo[1,5-a]pyrazine-3,5-dicarboxamide

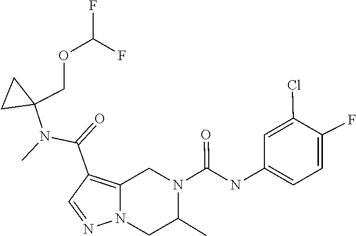

Rt (Method B) 3.37 mins, m/z 486/488 [M+H]+

¹H NMR (400 MHz, DMSO-d6) δ 9.02; (s, 1H), 7.85; (s, 1H), 7.74; (dd, J=6.9, 2.5 Hz, 1H), 7.45-7.38; (m, 1H), 7.31; (t, J=9.1 Hz, 1H), 6.71; (t, J=75.8 Hz, 1H), 5.35-5.18; (m, 1H), 4.94-4.82; (m, 1H), 4.44; (d, 1H), 4.24; (dd, J=12.8, 4.3 Hz, 1H), 4.17-3.95; (m, 3H), 3.22-2.90; (m, 3H), 1.12; (d, J=6.7 Hz, 3H), 0.95; (s, 4H).

### Example 7

N5-(3-chloro-4-fluorophenyl)-N3-methyl-N3-[1-(pyridin-4-yl)cyclopropyl]-4H,5H,6H,7H-pyrazolo[1,5-a]pyrazine-3,5-dicarboxamide

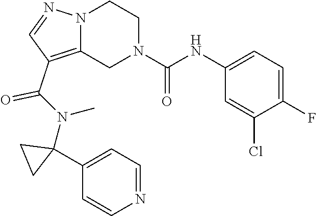 [SMILES: CN(C(=O)c1cnn2c1CN(C(=O)Nc1ccc(F)c(Cl)c1)CC2)C1(c2ccncc2)CC1]

Rt (Method A) 2.95 mins, m/z 469/471 [M+H]+

¹H NMR (400 MHz, DMSO-d6) δ 9.08; (s, 1H), 8.63-8.37; (m, 2H), 8.00-7.68; (m, 1H), 7.64-7.36; (m, 1H), 7.31; (t, J=9.1 Hz, 1H), 7.20-6.80; (m, 3H), 5.07-4.74; (m, 2H), 4.31-3.67; (m, 4H), 3.23-2.94; (m, 3H), 1.84-1.30; (m, 4H).

### Example 8

N5-(3-chloro-4-fluorophenyl)-N3-methyl-N3-[1-(pyrimidin-2-yl)cyclopropyl]-4H,5H,6H,7H-pyrazolo[1,5-a]pyrazine-3,5-dicarboxamide

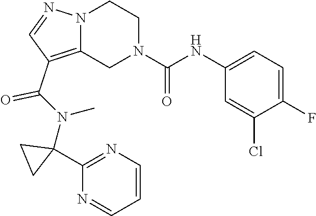 [SMILES: CN(C(=O)c1cnn2c1CN(C(=O)Nc1ccc(F)c(Cl)c1)CC2)C1(c2ncccn2)CC1]

Rt (Method A) 3 mins, m/z 470/472 [M+H]+

¹H NMR (400 MHz, DMSO-d6) δ 9.07; (s, 1H), 8.84-8.63; (m, 2H), 7.79-7.67; (m, 1H), 7.46-7.24; (m, 3H), 6.78; (s, 1H), 5.00-4.76; (m, 2H), 4.26-3.90; (m, 3H), 3.84-3.68; (m, 1H), 3.10; (s, 3H), 1.96-1.80; (m, 1H), 1.66-1.33; (m, 3H).

### Example 9

N5-(3-chloro-4-fluorophenyl)-N3-[1-(hydroxymethyl)cyclopropyl]-N3-methyl-4H,5H,6H,7H-pyrazolo[1,5-a]pyrazine-3,5-dicarboxamide

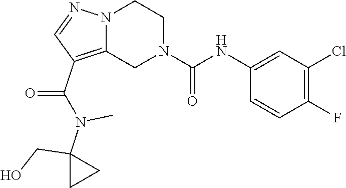 [SMILES: CN(C(=O)c1cnn2c1CN(C(=O)Nc1ccc(F)c(Cl)c1)CC2)C1(CO)CC1]

Rt (Method A) 2.77 mins, m/z 422/424 [M+H]+

¹H NMR (400 MHz, DMSO-d6) δ 9.07; (s, 1H), 8.17-7.76; (m, 1H), 7.73; (dd, J=6.8, 2.6 Hz, 1H), 7.41; (ddd, J=9.1, 4.4, 2.6 Hz, 1H), 7.31; (t, J=9.1 Hz, 1H), 5.32-4.92; (m, 1H), 4.92-4.72; (m, 2H), 4.24-4.12; (m, 2H), 4.12-3.73; (m, 2H), 3.72-3.53; (m, 2H), 3.22-2.88; (m, 3H), 1.21-0.60; (m, 4H).

### Example 10

N5-(3-chloro -4-fluorophenyl)-N3-(2-hydroxyethyl)-N3-[1-(hydroxymethyl)cyclopropyl]-4H,5H,6H,7H-pyrazol [1,5-a]pyrazine-3,5-dicarboxamide

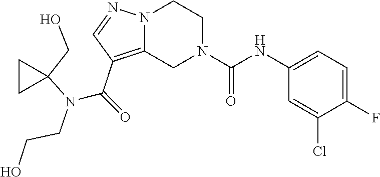 [SMILES: O=C(Nc1ccc(F)c(Cl)c1)N1CCn2ncc(C(=O)N(CCO)C3(CO)CC3)c2C1]

Rt (Method B) 2.72 mins, m/z 452/454 [M+H]+

¹H NMR (400 MHz, DMSO-d6) δ 9.08; (s, 1H), 7.92; (s, 1H), 7.73; (dd, J=6.8, 2.6 Hz, 1H), 7.41; (ddd, J=9.1, 4.4, 2.6 Hz, 1H), 7.31; (t, J=9.1 Hz, 1H), 5.18-5.01; (m, 1H), 4.94-4.73; (m, 3H), 4.22-3.39; (m, 10H), 1.39-0.61; (m, 4H).

### Example 11

N-(3-chloro-4-fluorophenyl) -3-{8-oxa-4-azaspiro[2.6]nonane-4-carbonyl}-4H,5H,6H,7H-pyrazolo[1,5-a]pyrazine-5-carboxamide

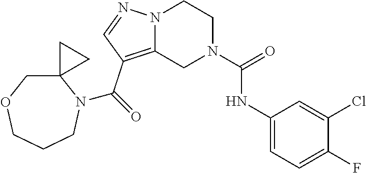 [SMILES: O=C(Nc1ccc(F)c(Cl)c1)N1CCn2ncc(C(=O)N3CCCOCC34CC4)c2C1]

Rt (Method B) 2.95 mins, m/z 448/450 [M+H]+

¹H NMR (400 MHz, DMSO-d6) δ 9.08; (s, 1H), 7.76-7.68; (m, 2H), 7.42; (ddd, J=9.1, 4.4, 2.7 Hz, 1H), 7.31; (t, J=9.1 Hz, 1H), 4.93-4.79; (m, 2H), 4.23-4.14; (m, 2H), 4.11-3.37; (m, 8H), 2.01-1.91; (m, 2H), 1.19-0.78; (m, 4H).

### Example 12

N-(3-chloro-4-fluorophenyl)-3-{7-oxa-4-azaspiro[2.6]nonane-4-carbonyl}-4H,5H,6H,7H-pyrazolo[1,5-a]pyrazine-5-carboxamide

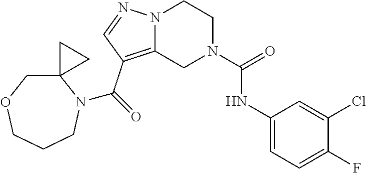 [SMILES: O=C(Nc1ccc(F)c(Cl)c1)N1CCn2ncc(C(=O)N3CCCOCC34CC4)c2C1]

Rt (Method B) 2.95 mins, m/z 448/450 [M+H]+

¹H NMR (400 MHz, DMSO-d6) δ 9.09; (s, 1H), 7.99-7.60; (m, 2H), 7.42; (ddd, J=9.1, 4.4, 2.7 Hz, 1H), 7.31; (t, J=9.1 Hz, 1H), 4.97-4.76; (m, 2H), 4.39-3.46; (m, 10H), 2.06-1.21; (m, 2H), 1.13-0.71; (m, 4H).

### Example 13

N5-(3-chloro-4-fluorophenyl)-N3-methyl-N3-(1-{[(2,2,2-trifluoroethyl)amino]methyl}cyclopropyl)-4H,5H,6H,7H-pyrazolo[1,5-a]pyrazine -3,5-dicarboxamide

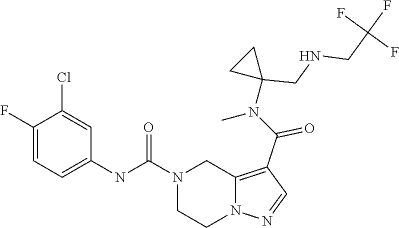

Rt (Method A) 3.22 mins, m/z 503/505 [M+H]+

¹H NMR (400 MHz, DMSO-d6) δ 9.09; (s, 1H), 8.04-7.65; (m, 2H), 7.49-7.38; (m, 1H), 7.31; (t, J=9.1 Hz, 1H), 4.99-4.72; (m, 2H), 4.28-3.66; (m, 4H), 3.30-2.55; (m, 8H), 1.33-0.61; (m, 4H).

### Example 14

N5-(3-chloro-4-fluorophenyl)-N3-{1-[2-(difluoromethoxy)ethyl]cyclobutyl}-N3-methyl-4H,5H,6H,7H-pyrazolo[1,5-a]pyrazine-3,5- dicarboxamide

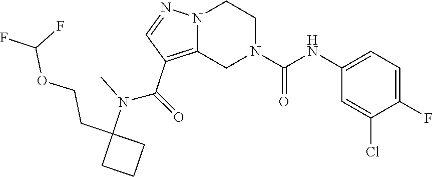 [SMILES: CN(C(=O)c1cnn2c1CN(C(=O)Nc1ccc(F)c(Cl)c1)CC2)C1(CCOC(F)F)CCC1]

Rt (Method A2) 3.86 mins, m/z 500/502 [M+H]+

### Example 15

N5-(3-chloro-4-fluorophenyl) -N3-{1-[2-(difluoromethoxy)ethyl]cyclopentyl}-N3-methyl-4H,5H,6H,7H -pyrazolo [1,5-a]pyrazine -3,5- dicarboxamide

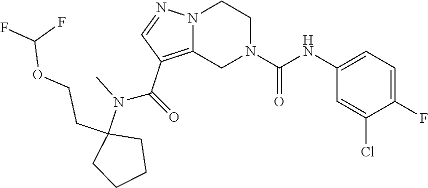 [SMILES: CN(C(=O)c1cnn2c1CN(C(=O)Nc1ccc(F)c(Cl)c1)CC2)C1(CCOC(F)F)CCCC1]

Rt (Method A2) 4.01 mins, m/z 514/516 [M+H]+

### Example 16

N5-(3-chloro-4-fluorophenyl)-N3-{4-[2-(difluoromethoxy)ethyl]oxan-4-yl}-N3-methyl-4H,5H,6H,7H-pyrazolo[1,5-a]pyrazine-3,5- dicarboxamide

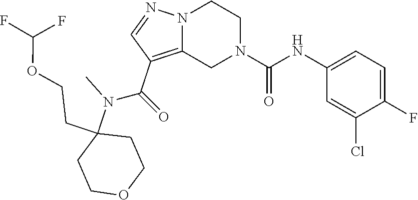 [SMILES: CN(C(=O)c1cnn2c1CN(C(=O)Nc1ccc(F)c(Cl)c1)CC2)C1(CCOC(F)F)CCOCC1]

Rt (Method A2) 3.58 mins, m/z 530/532 [M+H]+

### Example 17

N5-(3-chloro-4-fluorophenyl)-N3-cyclopropyl-N3,6-dimethyl-4H,5H,6H,7H -pyrazolo[1,5-a]pyrazine-3,5-dicarboxamide

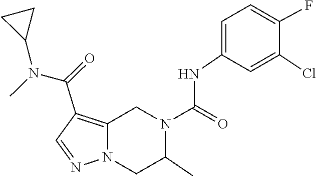 [SMILES: CC1Cn2ncc(C(=O)N(C)C3CC3)c2CN1C(=O)Nc1ccc(F)c(Cl)c1]

Rt (Method A) 3.11 mins, m/z 406/408 [M+H]+

¹H NMR (400 MHz, DMSO-d6) δ 8.99; (s, 1H), 8.00; (s, 1H), 7.74; (dd, J=6.9, 2.6 Hz, 1H), 7.46-7.39; (m, 1H), 7.31; (t, J=9.1 Hz, 1H), 5.25; (d, J=18.3 Hz, 1H), 4.93-4.84; (m, 1H), 4.46; (d, J=18.3 Hz, 1H), 4.25; (dd, J=12.9, 4.4 Hz, 1H), 4.14; (d, J=12.8 Hz, 1H), 3.13-3.04; (m, 1H), 2.96; (s, 3H), 1.12; (d, J=6.8 Hz, 3H), 0.84-0.75; (m, 2H), 0.69-0.51; (m, 2H).

### Biochemical Capsid Assembly Assay

The screening for assembly effector activity was done based on a fluorescence quenching assay published by Zlotnick et al. (2007). The C-terminal truncated core protein containing 149 amino acids of the N-terminal assembly domain fused to a unique cysteine residue at position 150 and was expressed in E. coli using the pET expression system (Merck Chemicals, Darmstadt). Purification of core dimer protein was performed using a sequence of size exclusion chromatography steps. In brief, the cell pellet from 1 L BL21 (DE3) Rosetta2 culture expressing the coding sequence of core protein cloned NdeI/XhoI into expression plasmid pET21b was treated for 1 h on ice with a native lysis buffer (Qproteome Bacterial Protein Prep Kit; Qiagen, Hilden). After a centrifugation step the supernatant was precipitated during 2 h stirring on ice with 0.23 g/ml of solid ammonium sulfate. Following further centrifugation the resulting pellet was resolved in buffer A (100 mM Tris, pH 7.5; 100 mM NaCl; 2 mM DTT) and was subsequently loaded onto a buffer A equilibrated CaptoCore 700 column (GE HealthCare, Frankfurt). The column flow through containing the assembled HBV capsid was dialyzed against buffer N (50 mM NaHCO3 pH 9.6; 5 mM DTT) before urea was added to a final concentration of 3M to dissociate the capsid into core dimers for 1.5 h on ice. The protein solution was then loaded onto a 1 L Sephacryl S300 column. After elution with buffer N core dimer containing fractions were identified by SDS-PAGE and subsequently pooled and dialyzed against 50 mM HEPES pH 7.5; 5 mM DTT. To improve the assembly capacity of the purified core dimers a second round of assembly and disassembly starting with the addition of 5 M NaCl and including the size exclusion chromatography steps described above was performed. From the last chromatography step core dimer containing fractions were pooled and stored in aliquots at concentrations between 1.5 to 2.0 mg/ml at −80° C.

Immediately before labelling the core protein was reduced by adding freshly prepared DTT in a final concentration of 20 mM. After 40 min incubation on ice storage buffer and DTT was removed using a Sephadex G-25 column (GE HealthCare, Frankfurt) and 50 mM HEPES, pH 7.5. For labelling 1.6 mg/ml core protein was incubated at 4° C. and darkness overnight with BODIPY-FL maleimide (Invitrogen, Karlsruhe) in a final concentration of 1 mM. After labelling the free dye was removed by an additional desalting step using a Sephadex G-25 column. Labelled core dimers were stored in aliquots at 4° C. In the dimeric state the fluorescence signal of the labelled core protein is high and is quenched during the assembly of the core dimers to high molecular capsid structures. The screening assay was performed in black 384 well microtiter plates in a total assay volume of 10 μl using 50 mM HEPES pH 7.5 and 1.0 to 2.0 μM labelled core protein. Each screening compound was added in 8 different concentrations using a 0.5 log-unit serial dilution starting at a final concentration of 100 μM, 31.6 μM or 10 μM, In any case the DMSO concentration over the entire microtiter plate was 0.5%. The assembly reaction was started by the injection of NaCl to a final concentration of 300 μM which induces the assembly process to approximately 25% of the maximal quenched signal. 6 min after starting the reaction the fluorescence signal was measured using a Clariostar plate reader (BMG Labtech, Ortenberg) with an excitation of 477 nm and an emission of 525 nm. As 100% and 0% assembly control HEPES buffer containing 2.5 M and 0 M NaCl was used. Experiments were performed thrice in triplicates. EC₅₀ values were calculated by non-linear regression analysis using the Graph Pad Prism 6 software (GraphPad Software, La Jolla, USA).

### Determination of HBV DNA from the Supernatants of HepAD38 Cells

The anti-HBV activity was analysed in the stable transfected cell line HepAD38, which has been described to secrete high levels of HBV virion particles (Ladner et al., 1997). In brief, HepAD38 cells were cultured at 37° C. at 5% CO₂ and 95% humidity in 200 μl maintenance medium, which was Dulbecco's modified Eagle's medium/Nutrient Mixture F-12 (Gibco, Karlsruhe), 10% fetal bovine serum (PAN Biotech Aidenbach) supplemented with 50 μg/ml penicillin/streptomycin (Gibco, Karlsruhe), 2 mM L-glutamine (PAN Biotech, Aidenbach), 400 μg/ml G418 (AppliChem, Darmstadt) and 0.3 μg/ml tetracycline. Cells were subcultured once a week in a 1:5 ratio, but were usually not passaged more than ten times. For the assay 60,000 cells were seeded in maintenance medium without any tetracycline into each well of a 96-well plate and treated with serial half-log dilutions of test compound. To minimize edge effects the outer 36 wells of the plate were not used but were filled with assay medium. On each assay plate six wells for the virus control (untreated HepAD38 cells) and six wells for the cell control (HepAD38 cells treated with 0.3 μg/ml tetracycline) were allocated, respectively. In addition, one plate set with reference inhibitors like BAY 41-4109, entecavir, and lamivudine instead of screening compounds were prepared in each experiment. In general, experiments were performed thrice in triplicates. At day 6 HBV DNA from 100 μl filtrated cell culture supernatant (AcroPrep Advance 96 Filter Plate, 0.45 μM Supor membran, PALL GmbH, Dreieich) was automatically purified on the MagNa Pure LC instrument using the MagNA Pure 96 DNA and Viral NA Small Volume Kit (Roche Diagnostics, Mannheim) according to the instructions of the manufacturer. EC50 values were calculated from relative copy numbers of HBV DNA In brief, 5 μl of the 100 μl eluate containing HBV DNA were subjected to PCR LC₄₈₀ Probes Master Kit (Roche) together with 1 μM antisense primer tgcagaggtgaagcgaagtgcaca, 0.5 μM sense primer gacgtcctttgtttacgtcccgtc, 0.3 μM hybprobes acggggcgcacctctctttacgcgg-FL and LC₆₄₀-ctccccgtctgtgccttctcatctgc-PH (TIBMolBiol, Berlin) to a final volume of 12.5 μl. The PCR was performed on the Light Cycler 480 real time system (Roche Diagnostics, Mannheim) using the following protocol: Pre-incubation for 1 min at 95° C., amplification: 40 cycles×(10 sec at 95° C., 50 sec at 60° C., 1 sec at 70° C.), cooling for 10 sec at 40° C. Viral load was quantitated against known standards using HBV plasmid DNA of pCH-9/3091 (Nassal et al., 1990, Cell 63: 1357-1363) and the LightCycler 480 SW 1.5 software (Roche Diagnostics, Mannheim) and EC₅₀ values were calculated using non-linear regression with GraphPad Prism 6 (GraphPad Software Inc., La Jolla, USA).

TABLE 1
Biochemical and antiviral activities

| Example | CC₅₀ (μM) | Cell Activity | Assembly Activity |
|---|---|---|---|
| Example 1 | >10 | +++ | A |
| Example 2 | >10 | +++ | A |
| Example 3 | >10 | +++ | A |
| Example 4 | >10 | +++ | A |
| Example 5 | >10 | +++ | A |
| Example 6 | >10 | +++ | A |
| Example 7 | >10 | +++ | A |
| Example 8 | >10 | +++ | A |
| Example 9 | >10 | +++ | A |
| Example 10 | >10 | +++ | A |
| Example 11 | >10 | +++ | A |
| Example 12 | >10 | +++ | A |
| Example 13 | >10 | +++ | A |
| Example 14 | >10 | +++ | A |
| Example 15 | >10 | +++ | A |
| Example 16 | >10 | +++ | A |
| Example 17 | >10 | +++ | A |

In Table 1, “+++” represents an EC₅₀ < 1 μM; “++” represents 1 μM < EC₅₀ < 10 μM; “+” represents EC₅₀ < 100 μM (Cell activity assay)
In Table 1, “A” represents an IC₅₀ < 5 μM; “B” represents 5 μM < IC₅₀ < 10 μM; “C” represents IC₅₀ < 100 μM (Assembly assay activity)

### Cell Viability Assay

Using the AlamarBlue viability assay cytotoxicity was evaluated in HepAD38 cells in the presence of 0.3 μg/ml tetracycline, which blocks the expression of the HBV genome. Assay condition and plate layout were in analogy to the anti-HBV assay, however other controls were used. On each assay plate six wells containing untreated HepAD38 cells were used as the 100% viability control, and six wells filled with assay medium only were used as 0% viability control. In addition, a geometric concentration series of cycloheximide starting at 60 μM final assay concentration was used as positive control in each experiment. After six days incubation period Alamar Blue Presto cell viability reagent (ThermoFisher, Dreieich) was added in 1/11 dilution to each well of the assay plate. After an incubation for 30 to 45 min at 37° C. the fluorescence signal, which is proportional to the number of living cells, was read using a Tecan Spectrafluor Plus plate reader with an excitation filter 550 nm and emission filter 595 nm, respectively. Data were normalized into percentages of the untreated control (100% viability) and assay medium (0% viability) before CC₅₀ values were calculated using non-linear regression and the GraphPad Prism 6.0 (GraphPad Software, La Jolla, USA). Mean EC₅₀ and CC₅₀ values were used to calculate the selectivity index (SI=CC₅₀/EC₅₀) for each test compound.

### In Vivo Efficacy Models

HBV research and preclinical testing of antiviral agents are limited by the narrow species- and tissue-tropism of the virus, the paucity of infection models available and the restrictions imposed by the use of chimpanzees, the only animals fully susceptible to HBV infection. Alternative animal models are based on the use of HBV-related hepadnaviruses and various antiviral compounds have been tested in woodchuck hepatitis virus (WHV) infected woodchucks or in duck hepatitis B virus (DHBV) infected ducks or in woolly monkey HBV (WM-HBV) infected tupaia (overview in Dandri et al., 2017, Best Pract Res Clin Gastroenterol 31, 273-279). However, the use of surrogate viruses has several limitations. For example is the sequence homology between the most distantly related DHBV and HBV is only about 40% and that is why core protein assembly modifiers of the HAP family appeared inactive on DHBV and WHV but efficiently suppressed HBV (Campagna et al., 2013, J. Virol. 87, 6931-6942). Mice are not HBV permissive but major efforts have focused on the development of mouse models of HBV replication and infection, such as the generation of mice transgenic for the human HBV (HBV tg mice), the hydrodynamic injection (HDI) of HBV genomes in mice or the generation of mice having humanized livers and/or humanized immune systems and the intravenous injection of viral vectors based on adenoviruses containing HBV genomes (Ad-HBV) or the adenoassociated virus (AAV-HBV) into immune competent mice (overview in Dandri et al., 2017, Best Pract Res Clin Gastroenterol 31, 273-279). Using mice transgenic for the full HBV genome the ability of murine hepatocytes to produce infectious HBV virions could be demonstrated (Guidotti et al., 1995, J. Virol., 69: 6158-6169). Since transgenic mice are immunological tolerant to viral proteins and no liver injury was observed in HBV-producing mice, these studies demonstrated that HBV itself is not cytopathic. HBV transgenic mice have been employed to test the efficacy of several anti-HBV agents like the polymerase inhibitors and core protein assembly modifiers (Weber et al., 2002, Antiviral Research 54 69-78; Julander et al., 2003, Antivir. Res., 59: 155-161), thus proving that HBV transgenic mice are well suitable for many type of preclinical antiviral testing in vivo.

As described in Paulsen et al., 2015, PLOSone, 10: e0144383 HBV-transgenic mice (Tg [HBV1.3 fsX⁻3′5′]) carrying a frameshift mutation (GC) at position 2916/2917 could be used to demonstrate antiviral activity of core protein assembly modifiers in vivo. In brief, The HBV-transgenic mice were checked for HBV-specific DNA in the serum by qPCR prior to the experiments (see section “Determination of HBV DNA from the supernatants of HepAD38 cells”). Each treatment group consisted of five male and five female animals approximately 10 weeks age with a titer of 10⁷-10⁸ virions per ml serum. Compounds were formulated as a suspension in a suitable vehicle such as 2% DMSO/98% tylose (0.5% Methylcellulose/99.5% PBS) or 50% PEG400 and administered per os to the animals one to three times/day for a 10 day period. The vehicle served as negative control, whereas 1 μg/kg entecavir in a suitable vehicle was the positive control. Blood was obtained by retro bulbar blood sampling using an Isoflurane Vaporizer. For collection of terminal heart puncture six hours after the last treatment blood or organs, mice were anaesthetized with isoflurane and subsequently sacrificed by CO₂ exposure. Retro bulbar (100-150 μl) and heart puncture (400-500 μl) blood samples were collected into a Microvette 300 LH or Microvette 500 LH, respectively, followed by separation of plasma via centrifugation (10 min, 2000 g, 4° C.). Liver tissue was taken and snap frozen in liquid N₂. All samples were stored at −80° C. until further use. Viral DNA was extracted from 50 μl plasma or 25 mg liver tissue and eluted in 50 μl AE buffer (plasma) using the DNeasy 96 Blood & Tissue Kit (Qiagen, Hilden) or 320 μl AE buffer (liver tissue) using the DNeasy Tissue Kit (Qiagen, Hilden) according to the manufacturer's instructions. Eluted viral DNA was subjected to qPCR using the LightCycler 480 Probes Master PCR kit (Roche, Mannheim) according to the manufacturer's instructions to determine the HBV copy number. HBV specific primers used included the forward primer 5′-CTG TAC CAA ACC TTC GGA CGG-3′, the reverse primer 5′-AGG AGA AAC GGG CTG AGG C-3′ and the FAM labelled probe FAM-CCA TCA TCC TGG GCT TTC GGA AAA TT-BBQ. One PCR reaction sample with a total volume of 20 μl contained 5 μl DNA eluate and 15 μl master mix (comprising 0.3 μM of the forward primer, 0.3 μM of the reverse primer, 0.15 μM of the FAM labelled probe). qPCR was carried out on the Roche LightCycler1480 using the following protocol: Pre-incubation for 1 min at 95° C., amplification: (10 sec at 95° C., 50 sec at 60° C., 1 sec at 70° C.)×45 cycles, cooling for 10 sec at 40° C. Standard curves were generated as described above. All samples were tested in duplicate. The detection limit of the assay is ˜50 HBV DNA copies (using standards ranging from 250-2.5×107 copy numbers). Results are expressed as HBV DNA copies/10 μl plasma or HBV DNA copies/100 ng total liver DNA (normalized to negative control).

It has been shown in multiple studies that not only transgenic mice are a suitable model to proof the antiviral activity of new chemical entities in vivo the use of hydrodynamic injection of HBV genomes in mice as well as the use of immune deficient human liver chimeric mice infected with HBV positive patient serum have also frequently used to profile drugs targeting HBV (Li et al., 2016, Hepat. Mon. 16: e34420; Qiu et al., 2016, J. Med. Chem. 59: 7651-7666; Lutgehetmann et al., 2011, Gastroenterology, 140: 2074-2083). In addition chronic HBV infection has also been successfully established in immunecompetent mice by inoculating low doses of adenovirus—(Huang et al., 2012, Gastroenterology 142: 1447-1450) or adeno-associated virus (AAV) vectors containing the HBV genome (Dion et al., 2013, J Virol. 87: 5554-5563). This models could also be used to demonstrate the in vivo antiviral activity of novel anti-HBV agents.

1. Compound of Formula II 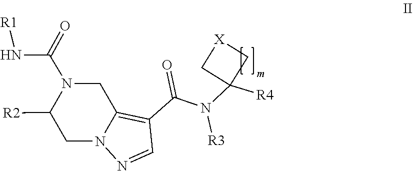
in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R3 is C1-C4 alkyl said C1-C4-alkyl is unsubstituted or substituted once, twice, or thrice with deuterium, OH or halo
R4 is selected from the group comprising C1-C2-alkyl-O-C1-C4-alkyl, C1-C2-hydroxyalkyl, C₁-C2-alkyl-O-C1-C4-haloalkyl, C1-C2-alkyl-NH-C1-C4-haloalkyl, C1-C2-alkyl-O-C3-C6-cycloalkyl, C1-C2-alkyl-S-C1-C4-alkyl, C1-C2-alkyl-SO₂-C1-C4-alkyl, C1-C2-alkyl-C≡N, C₁-C2-alkyl-C3-C7-heterocycloalkyl, C1-C2-alkyl-O—C(═O)(C3-C7-cycloalkyl)NH₂, C1-C2-alkyl-O—C(═O)(C1-C6-alkyl)NH₂, aryl and heteroaryl, wherein aryl or heteroaryl are optionally substituted once, twice or thrice with halo or C1-C6 alkyl
R3 and R4 are optionally connected to form a five, six or seven membered heterocyclic ring, wherein said heterocyclic ring is unsubstituted or substituted once, twice or thrice with halo, OH, carboxy, OCF₃, OCHF₂ or C≡N
X is O, CH₂, or NR5
m is 0, 1, 2 or 3
R5 is H or C1-C4-alkyl
or a pharmaceutically acceptable salt thereof
or a solvate or a hydrate of a compound of Formula II or the pharmaceutically acceptable salt thereof
or a prodrug of a compound of Formula II or a pharmaceutically acceptable salt or a solvate or a hydrate thereof.

2. A compound of Formula II according to claim 1 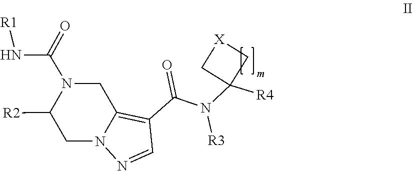
in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R3 is C1-C4 alkyl said C1-C4-alkyl is unsubstituted or substituted once, twice, or thrice with deuterium or halo
R4 is selected from the group comprising C1-C2-alkyl-O-C1-C4-alkyl, C1-C2-hydroxyalkyl, C1-C2-alkyl-O-C1-C4-haloalkyl, C₁-C2-alkyl-O-C3-C6-cycloalkyl, C1-C2-alkyl-S-C1-C4-alkyl, C1-C2-alkyl-SO₂-C1-C4-alkyl, C1-C2-alkyl-C≡N, C1-C2-alkyl-C3-C7-heterocycloalkyl, C1-C2-alkyl-O—C(═O)(C3-C7-cycloalkyl)NH₂, C1-C2-alkyl-O—C(═O)(C1-C6-alkyl)NH₂, aryl and heteroaryl, wherein aryl or heteroaryl are optionally substituted once, twice or thrice with halo or C1-C6 alkyl
R3 and R4 are optionally connected to form a five, six or seven membered heterocyclic ring, wherein said heterocyclic ring is unsubstituted or substituted once, twice or thrice with halo, OH, carboxy, OCF₃, OCHF₂ or C≡N
X is O, CH₂, or NR5
m is 0, 1 or 2
R5 is H or C1-C4-alkyl
or a pharmaceutically acceptable salt thereof
or a solvate or a hydrate of a compound of Formula II or the pharmaceutically acceptable salt thereof
or a prodrug of a compound of Formula II or a pharmaceutically acceptable salt or a solvate or a hydrate thereof.

3. A compound of Formula II according to claim 1, wherein aryl is C6-aryl, and/or heteroaryl is C1-C9 heteroaryl and wherein heteroaryl and heterocycloalkyl each has 1 to 4 heteroatoms each independently selected from N, O and S,
or a pharmaceutically acceptable salt thereof
or a solvate or a hydrate of a compound of Formula II or the pharmaceutically acceptable salt thereof
or a prodrug of a compound of Formula II or a pharmaceutically acceptable salt or a solvate or a hydrate thereof.

4. A compound of Formula II according to claim 1
or a pharmaceutically acceptable salt thereof
or a solvate or a hydrate of a compound of Formula II or the pharmaceutically acceptable salt thereof
or a prodrug of a compound of Formula II or a pharmaceutically acceptable salt or a solvate or a hydrate thereof,
wherein the prodrug is selected from the group comprising esters, carbonates, acetyloxy derivatives, amino acid derivatives and phosphoramidate derivatives.

5. A compound according to or claim 1 or a pharmaceutically acceptable salt thereof
or a solvate or a hydrate of said compound or the pharmaceutically acceptable salt thereof
or a prodrug of said compound or a pharmaceutically acceptable salt or a solvate or a hydrate thereof for addition to a pharmaceutical composition for use in the prevention or treatment of an HBV infection in subject.

6. A pharmaceutical composition comprising a compound of Formula II according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof, together with a pharmaceutically acceptable carrier.

7. A method of treating an HBV infection in an individual in need thereof, comprising administering to the individual a therapeutically effective amount of a compound of Formula II according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof.

8. A method for the preparation of a compound of Formula II according to claim 1 by reacting a compound of Formula III
R1—N═C═O III
in which R1 is as defined in claim 1, with a compound of Formula IV 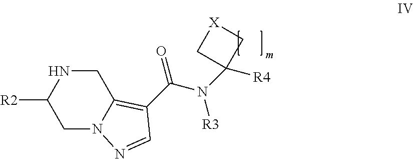
in which R2, R3, R4, X and m are as defined in claim 1.


SEQUENCE LISTING (7 sequences)

SEQ ID NO: 1 - DNA, 21 bp, Hepatitis B virus
ctgtaccaaaccttcggacgg

SEQ ID NO: 2 - DNA, 18 bp, Hepatitis B virus
aggagaaacgggctgagg

SEQ ID NO: 3 - DNA, 26 bp, Hepatitis B virus
ccatcatcctgggctttcggaaaatt

SEQ ID NO: 4 - DNA, 24 bp, Hepatitis B virus
tgcagaggtgaagcgaagtgcaca

SEQ ID NO: 5 - DNA, 24 bp, Hepatitis B virus
gacgtcctttgtttacgtcccgtc

SEQ ID NO: 6 - DNA, 25 bp, Hepatitis B virus
acggggcgcacctctctttacgcgg

SEQ ID NO: 7 - DNA, 26 bp, Hepatitis B virus
ctccccgtctgtgccttctcatctgc